(19) United States
(12) Patent Application Publication
(10) Pub. No.: US 2016/0184230 A1
(43) Pub. Date: Jun. 30, 2016

(54) MELT-PROCESSED POLYMERIC CELLULAR DOSAGE FORM

(71) Applicant: Aron H. BLAESI, Cambridge, MA (US)

(72) Inventors: ARON H. BLAESI, Cambridge, MA (US); NANNAJI SAKA, Cambridge, MA (US)

(73) Assignee: ARON H. BLAESI, Cambridge, MA (US)

(21) Appl. No.: 14/907,891

(22) PCT Filed: Apr. 30, 2015
(86) PCT No.: PCT/US15/28655

§ 371 Date: Jan. 27, 2016

Related U.S. Application Data
(60) Provisional application No. 61/986,262, filed on Apr. 30, 2014.

(51) Int. Cl. A61K 9/16; A61K 31/167

(52) U.S. Cl. CPC A61K 9/1641; A61K 31/167

(57) ABSTRACT

Presented herein are polymeric cellular dosage forms exhibiting improved immediate release properties, while maintaining high uniformity and satisfactory mechanical properties (e.g., to permit necessary handling). An exfoliating polymeric cellular dosage form is described herein that can be cost-effectively manufactured via batch or even non-batch (continuous or semi-continuous) melt processing. The solid dosage forms have a unique cellular microstructure featuring a number of open, interconnected cells. The cell walls contain the active ingredient(s) as well as an excipient that swells in the presence of a physiological fluid such as gastrointestinal fluid and/or saliva under physiological conditions.

### CROSS-REFERENCE TO RELATED APPLICATION

This application claims priority to and the benefit of, and incorporates herein by reference in its entirety, U.S. Provisional Patent Application No. 61/986,262, filed Apr. 30, 2014.

### FIELD OF THE INVENTION

This invention relates generally to microstructures, compositions and methods for immediate drug release. More particularly, in certain embodiments, the invention relates to cellular dosage forms.

### BACKGROUND OF THE INVENTION

Pharmaceutical dosage forms are formulations of biologically active drug substances and drug carriers or excipients. They can be solids, ranging from a few nanometers to several millimeters in size, semi-solids (e.g., ointments), liquids, or gases. For decades, the most prevalent dosage forms have been solids, particularly immediate-release oral tablets and capsules. Typically, they consist of a granular material structure compounded by blending and compacting drug and excipient particles.

Microstructures and solid-state properties of dosage forms are critical, determining the rate of drug release in the gastrointestinal tract and the concentration profile of drug at biological targets. After ingestion, the granular immediate-release dosage form is percolated by gastric fluid. The bonds between the particles are severed, resulting in disintegration of the dosage form into its particulate constituents.

Manufacturing the granular dosage forms, however, presents several problems. The process typically entails resource-intensive and time-consuming batch processing, for example, mixing, granulating, drying, milling, and screening followed by tableting and coating. Mixing and compacting the drug and excipient particles are hampered by particle segregation. Aggregates that exhibit poor dissolution properties may be formed during the process. Furthermore, the theoretical understanding of the physical behavior of granular media is incomplete. This limits opportunities for optimization of products and processes for their manufacture, particularly in areas related to the optimization of process control, the time and resources required in product and process development, and the time and resources required in manufacturing scale-up. Moreover, unacceptable batch-to-batch variations are not uncommon in drug dosage form manufacturing, resulting in out-of-specification product waste and expensive quality control.

Manufacturing dosage forms by casting or molding may mitigate many limitations. The material is fluidized either by a solvent or by melting and is handled in liquid form, thus imparting reproducible, predictive microstructure and properties. Several studies have shown, however, that cast dosage forms, particularly if they consist of biologically inert and chemically and physically stable polymeric excipients, are appropriate only for long-term or sustained release. They are not suitable for immediate drug release, as cast matrices resist percolation of the dissolution medium, giving a slow rate of drug release. Although the drug release rate of dosage forms based on solid matrices could be increased by adding substantial amounts of either highly soluble small molecules (e.g., specific types of sugars or polyols) or effervescent agents (e.g., sodium bicarbonate) to the formulation, the addition of such materials is typically inferior because such materials are bioactive and/or impair the stability of the dosage form.

There is therefore a need for polymeric solid dosage forms with improved immediate-release properties and uniform ingredient content which can be prepared by a cost-effective, predictable process.

### SUMMARY OF THE INVENTION

Presented herein are polymeric cellular dosage forms exhibiting improved immediate-release properties, while maintaining high uniformity and satisfactory mechanical properties (e.g., to permit necessary handling). An exfoliating polymeric cellular dosage form is described herein that can be cost-effectively manufactured via batch or even non-batch (continuous or semi-continuous) melt processing. The polymeric cellular dosage forms have a unique cellular microstructure featuring a number of open, interconnected cells. The cell walls contain the active ingredient(s) as well as an excipient that swells in the presence of a physiological fluid such as gastrointestinal fluid and/or saliva under physiological conditions.

Without wishing to be bound to any particular theory, it is believed that the presence of certain channels having two or more openings of different size allows initial percolation of the physiological fluid by capillary pressure differences, followed by penetration of the fluid into the cell walls, softening of the cell walls due to the penetrated excipient, rupture of the cell walls due to capillary pressure, rupture of cell walls due to differences in the density of fragments of the dosage form compared with the density of the dissolution fluid (e.g., rupture of walls due to buoyancy of a fragment, and rupture of walls due to gravity), rupture of cell walls due to the application of shear forces, or rupture of cell walls due to imbalances in the hydrostatic pressure in the dissolution medium. The ruptured cell walls may exfoliate as fragments from the structure and, together with the original structure, release drug into the dissolution medium. The surface area-to-volume ratio of the solid content is increased due to the exfoliation; thus, exfoliation of the structure speeds up drug release. The dosage form presented herein has a structure and material of the cell walls to promote exfoliation of fragments of the solid into the dissolution medium (physiological fluid), speeding drug release from the polymeric cellular dosage form.

The solid dosage form can be melt manufactured, e.g., via extrusion (or other form of mixing) and injection molding, with injection of a gas and/or supercritical fluid (e.g., nitrogen or carbon dioxide) to form the desired microstructure.

Thus, in one aspect, the invention is directed to a pharmaceutical solid dosage form (e.g., an oral tablet or capsule) comprising one or more hydrophilic excipients and one or more active ingredients, wherein the dosage form has a cellular microstructure with a plurality of cells (e.g., voids of substantially convex shape filled with a gas that is non-reactive with the active ingredients and the excipients, e.g., N₂, CO₂, and/or air), having walls comprising the one or more active ingredients and the one or more excipients (e.g., the one or more active ingredients embedded in the one or more excipients), wherein: (a) a fraction of the total number of cells in the solid dosage form are part of a cluster of two or more interconnected cells, said fraction in a range from 0.3 to 1 (e.g., 0.35 to 1, 0.4 to 1, or 0.45 to 1); (b) the cells have average size (e.g., average channel width, and/or average internal diameter) in a range from 5 μm to 1200 μm (e.g., from 5 μm to 1000 μm, 10 μm to 1000 μm); (c) the cells have average wall thickness, h₀, in a range from 1 μm to 500 μm (e.g., from 1 μm to 300 μm, 3 μm to 300 μm); (d) the solid dosage form has void volume fraction with respect to total volume, φᵥ, in a range from 0.2 to 0.85 (e.g., from 0.3 to 0.8, from 0.35 to 0.75, no less than 0.3, no less than 0.35, or no less than about 0.4); and (e) the solid dosage form has at least one dimension (e.g., length, width, and/or thickness) greater than 1 mm. In certain embodiments, the fraction of total cells that are part of a cluster of interconnected cells is on the low end of the scale (e.g., from 0.3 to 0.4) where the excipient is highly soluble and/or has low molecular weight (e.g., PEG 8000), and, in other embodiments, the fraction of total cells that are part of a cluster of interconnected cells is on the higher end of the scale (e.g., 0.8 to 1) wherein the excipient is less soluble and/or has a high molecular weight.

In certain embodiments, standard deviation of the cell size (e.g., among all the cells in the solid dosage form) is less than the average cell size in the solid dosage form (e.g. where the average cell size is smaller than 100 μm) (e.g., and wherein standard deviation of the cell size is less than half the average cell size where the average cell size is within a range from 100 μm to 1200 μm). In certain embodiments, standard deviation of the cell wall thickness (e.g., among all the cell walls in the solid dosage form) is less than the average cell wall thickness.

In certain embodiments, the one or more excipients is/are absorptive of a physiological fluid (e.g., water, saline, saliva, and/or gastrointestinal fluid) under physiological conditions (e.g., at about 37° C., e.g., upon ingestion by a subject) when the one or more excipients is/are exposed to the physiological fluid (e.g., and wherein rate of penetration of the physiological fluid into the solid dosage form (e.g., velocity of the penetrating front of the physiological fluid) is greater than about h₀/1800 μm/s (e.g., greater than about h₀/300 μm/s, greater than h₀/150)). In certain embodiments, the solid dosage form has a composition and structure such that effective diffusion coefficient of the physiological fluid into the solid (i.e., the cell wall) is no less than 1·10⁻¹¹ m²/s (e.g., less than 3·10⁻¹¹ m²/s, no less than 6·10⁻¹¹ m²/s, or no less than 9·10⁻¹¹ m²/s).

In certain embodiments, shear viscosity of the one or more excipients (e.g., individually and/or in their totality where there is more than one excipient) is no greater than about 100 Pa·s (e.g., no greater than 50 Pa·s, or no greater than 25 Pa·s) upon absorption of (e.g., saturation with) a physiological fluid (e.g., water, saline, saliva, and/or gastrointestinal fluid).

In certain embodiments, solubility of the excipient in a physiological fluid (e.g., water, saline, saliva, and/or gastrointestinal fluid) is no less than about 1 g/l (e.g. no less than 10 g/l, no less than 30 g/l, or no less than 50 g/l). For example, PEG has a solubility of about 500 g/l.

In certain embodiments, tensile strength of the dosage form is no less than about 0.05 N/mm² (e.g., no less than about 0.15 N/mm², no less than about 0.25 N/mm², or no less than about 0.3 N/mm²).

In certain embodiments, the one or more excipients comprises a polymer having weight average molecular weight in a range from 1,000 g/mol to 300,000 g/mol (e.g., from 2000 g/mol to 200,000 g/mol, or from 2000 g/mol to 150,000 g/mol). In certain embodiments, the one or more excipients comprises polyethylene glycol having weight average molecular weight in a range from 4,000 g/mol to 100,000 g/mol (e.g., PEG 6000 to PEG 90,000, or PEG 8000 to PEG 70,000, particularly where PEG is the sole or primary (>80%) excipient).

In certain embodiments, the walls of the dosage form are composed of a solid having void volume fraction no greater than about 0.1 (e.g., no greater than about 0.05; e.g., a substantially non-porous solid).

In certain embodiments, the walls of the dosage form have an excipient volume fraction, with respect to total wall volume, greater than 0.12.

In certain embodiments, the dosage form further comprises one or more fast eroding excipients (e.g., sucrose, sorbitol, xylitol, dextrose, maltitol, and/or lactitol) (e.g., wherein each of the one or more fast eroding excipients has a characteristic erosion rate (ψ=(solubility×diffusivity^{1/2})/(π^{1/2}×density)) greater than about 5×10⁻⁵ m/s^{1/2} upon ingestion by the subject), wherein φₑ, volume fraction of the fast eroding excipient(s) with respect to the total wall volume, is within a range from about 0.03 to about 0.4 (e.g., about 0.03 to about 0.35, or about 0.05 to 0.35). In certain embodiments, the dosage form further comprises one or more effervescent agents (e.g., sodium bicarbonate), wherein φₑ, volume fraction of the effervescent agent(s) with respect to total wall volume, is within a range from about 0.03 to about 0.4 (e.g., about 0.03 to about 0.35, or about 0.05 to about 0.35). In certain embodiments, the dosage form further comprises one or more fillers, one or more stabilizers, one or more preservatives, one or more taste maskers, one or more colorants, or any combination thereof.

In certain embodiments, solid drug contents of the dosage form are converted into molecularly dissolved units in less than about 30 minutes (e.g., less than about 25 minutes, 20 minutes, 15 minutes, 10 minutes, or 5 minutes) after ingestion.

In another aspect, the invention is directed to a method of manufacturing a pharmaceutical cellular dosage form (e.g., an oral tablet), the method comprising: (a) mixing (i) and (ii) with application of shear force (e.g., via extrusion): (i) one or more excipients (e.g., each of the excipients or the excipient composite having a melting temperature or a glass transition temperature within a range from about 35° C. to about 195° C., e.g., from 40° C. to 190° C.) (e.g., wherein the excipient(s) is/are thermoplastic and transition(s) from solid or solid-like to fluid or fluid-like at a temperature within a range from about 35° C. to about 195° C., e.g., from 40° C. to 190° C.), (ii) one or more pharmaceutically active ingredients (e.g., acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator); (b) introducing a foaming agent (e.g., a gas (e.g., nitrogen and CO₂) and/or a supercritical fluid under pressure, e.g., wherein the pressure is about 2 MPa to about 30 MPa (e.g., from about 3 MPa to about 25 MPa)) into the mixture (e.g., wherein the mixture is at a temperature between about 40° C. and about 200° C. when the foaming agent is introduced, e.g., wherein the mixture has transitioned from solid or solid-like to fluid or fluid-like upon introduction of the foaming agent); and (c) introducing the mixture into a mold (e.g., via mold injection) (e.g., wherein the injected volume of the mixture is less than the mold capacity), such that the pharmaceutical cellular dosage form produced thereby has a cellular microstructure with a plurality of cells (e.g., voids of substantially convex shape filled with a gas that is non-reactive with the active ingredients and the excipients, e.g., N₂, CO₂, and/or air), having walls comprising the one or more active ingredients and the one or more excipients (e.g., the one or more active ingredients embedded in the one or more excipients), wherein one, two, three, four, or all five of items (A) through (E) apply: (A) a fraction of the total number of cells in the solid dosage form are part of a cluster of two or more interconnected cells, said fraction in a range from 0.3 to 1 (e.g., 0.35 to 1, 0.4 to 1, or 0.45 to 1); (B) the cells have average size (e.g., average channel width, and/or average internal diameter) in a range from 5 μm to 1200 μm (e.g., from 5 μm to 1000 μm, or from 10 μm to 1000 μm); (C) the cells have average wall thickness, h₀, in a range from 1 μm to 500 μm (e.g., from 1 μm to 300 μm, or from 3 μm to 300 μm); (D) the solid dosage form has void volume fraction with respect to total volume, φᵥ, in a range from 0.2 to 0.85 (e.g., from 0.3 to 0.8, from 0.35 to 0.75, no less than 0.3, no less than 0.35, or no less than about 0.4); and (E) the solid dosage form has at least one dimension (e.g., length, width, and/or thickness) greater than 1 mm.

In certain embodiments, the one or more excipients comprises a polyethylene glycol with molecular weight above 1500 g/mol—e.g., PEG 8000, PEG 12000, PEG 20000, PEG 35000, PEG below 100,000 Da, PEG below 75,000 Da, PEG below 50,000 Da—a poloxamer (e.g. poloxamer 188 or poloxamer 407), a polymethacrylate, a polyvinylpyrrolidones (e.g. 1-vinyl-2-pyrrolidinone polymer (Povidone) or polyvinylpyrrolidone-vinyl acetate copolymer (Copovidone)), Kollicoat IR, glyceryl behenate, glyceryl distearate, and/or a stearic acid.

In certain embodiments, the method further comprises dissolving the foaming agent in the mixture so that the concentration of the foaming agent is homogeneous in the mixture (e.g., under shear force).

In certain embodiments, the method further comprises reducing the pressure of the mixture (e.g., at a partial pressure of the foaming agent in the mixture between 2 MPa to 30 MPa (e.g., between 3 MPa and 25 MPa)) (e.g., at a temperature within a range from about 40° C. to about 200° C. and in a time of about 0.01 s to about 5 mins (e.g., about 0.01 s to about 3 mins), or at a temperature within a range from about 45° C. to about 190° C. and in a time of about 0.03 s to about 3 mins) so that the foaming agent is supersaturated in the mixture and gas bubbles nucleate and grow. In certain embodiments, the method further comprises reducing the temperature of the mixture so that the mixture solidifies as the cellular dosage forms.

In certain embodiments, the method further comprises introducing a coating material in the mold or applying the coating material directly to the dosage form.

Elements of embodiments described with respect to one aspect of the invention can be applied with respect to another aspect. For example, certain embodiments of the method claims can include features of the composition claims, and vice versa.

### BRIEF DESCRIPTION OF THE DRAWINGS

The foregoing and other objects, aspects, features, and advantages of the present disclosure will become more apparent and better understood by referring to the following description taken in conduction with the accompanying drawings, in which:

FIGS. 1A-1J are scanning electron microscope (SEM) images of exemplary melt-processed cellular dosage forms.

FIG. 1A shows SEM image of cast specimen with polyethylene glycol (PEG) 8k (control). (Process A)

FIG. 1B shows a cellular dosage form with PEG 8k processed at soaking temperature, Tₛ=70° C., soaking pressure, pₛ=4.1 MPa, pressure release time, τᵣ=3 s. (Process B)

FIG. 1C shows a cellular dosage form with PEG 8k processed at Tₛ=110° C., pₛ=5.5 MPa, τᵣ=3 s. (Process C)

FIG. 1D shows a cellular dosage form with PEG 8k processed at Tₛ=130° C., pₛ=4.1 MPa, τᵣ=1 min. (Process D)

FIG. 1E shows a cellular dosage form with PEG 8k processed at Tₛ=130° C., pₛ=6.2 MPa, τᵣ=3 s. (Process E)

FIG. 1F shows a cellular dosage form with PEG 8k processed at Tₛ=130° C., pₛ=6.9 MPa, τᵣ=1 min. (Process F)

FIG. 1G shows a cellular dosage form with PEG 12k processed at Tₛ=130° C., pₛ=8.2 MPa, τᵣ=3 s.

FIG. 1H shows a cellular dosage form with PEG 20k processed at Tₛ=130° C., pₛ=8.2 MPa, τᵣ=3 s.

FIG. 1I shows a cellular dosage form with PEG 35k processed at Tₛ=130° C., pₛ=8.2 MPa, τᵣ=3 s.

FIG. 1J shows a cellular dosage form with PEO 100k processed at Tₛ=130° C., pₛ=8.2 MPa, τᵣ=3 s.

FIG. 2 are snapshots of closed-cell and open-cell dosage forms during dissolution. The excipient was PEG 8000 and the drug was Acetaminophen at a weight fraction equal to 0.6. Top row shows closed-cell dosage form with φ_{y}=0.2 prepared by Process B. Bottom row shows open-cell dosage form with φ_{y}=0.55 prepared by Process E. The samples were attached to either a ring or posts using glue. After immersion of the samples into the dissolution medium, images were taken continuously with a conventional photocamera or a high speed camera.

FIG. 3A depicts dissolution curves of selected dosage forms with adapted paddle tests. The amount of drug dissolved in the dissolution medium was measured versus time. The excipient was PEG 8000 and the drug was Acetaminophen at a weight fraction equal to 0.6.

FIG. 3B is a graph of dissolved drug amount as a function of time. The volume fraction of voids was 0.55. The dosage forms were processed at Tₛ=130° C., pₛ=8.2 MPa, tᵣ=3 s. The drug was Acetaminophen at a weight fraction equal to 0.6.

FIG. 3C depicts drug release flux, j_{d}, of the cellular dosage forms versus volume fraction of voids. Drug release fluxes are obtained by dividing 80% of the drug content (196 mg) with t_{0.8} (Table 1) and the projected surface area of the dosage form (132.73 mm²). The excipient was PEG 8000 and the drug was Acetaminophen at a weight fraction equal to 0.6. If the drug particles dissolve rapidly once they are released from the dosage form, then the drug release flux is equal to the flux of the eroding excipient divided by the excipient volume fraction multiplied by the drug volume fraction. The dashed line represents an exponential fit of the data. The letters A-F indicate the process designation from FIGS. 1A-1F.

FIGS. 3D-3F show graphs of drug release flux. The drug release flux was calculated with the drug content in the dosage form, the time to dissolve 80 percent of the drug content, and the projected surface area of the dosage form.

FIG. 3D shows drug release flux as a function of void volume fraction.

FIGS. 3E and 3F show drug release flux as a function of excipient molecular weight using polyethylene glycols and polyethylene oxides as excipient.

FIGS. 4A-4D illustrate schematics of cellular dosage forms and their dissolution mechanisms. The drug is embedded in the structure as particles dispersed in the excipient matrix.

FIG. 4A shows a non-porous cell structure with surface erosion of the excipient as dominant dissolution mechanism.

FIG. 4B shows a closed-cell structure with increased surface area for erosion.

FIG. 4C shows a partially interconnected cell structure with dissolution medium capable of percolating part of the voids.

FIG. 4D shows an open-cell structure percolated by the dissolution medium and with a remainder of entrapped air in a subset of the cells.

FIG. 4E illustrates an exemplary percolation process in cellular dosage forms.

FIGS. 5A-5C depict mechanical properties of the selected cellular dosage forms from diametral compression tests. The excipient was PEG 8000 and the drug was Acetaminophen at a weight fraction equal to 0.6.

FIG. 5A shows a graph showing the effect of displacement on compressive force.

FIG. 5B is a graph showing the effect of volume fraction of voids on tensile strength. Tensile strength of a dosage form is obtained from the applied force on a disk specimen during/before fracture. The dashed line represents a linear fit of the data. The letters A-F indicate the process designation.

FIG. 5C shows fractured dosage forms due to applied mechanical forces. (unfoamed (left), process B (middle), Process F (right)).

FIGS. 5D and 5E depict mechanical properties of cellular dosage forms with certain excipient molecular weights. The drug was Acetaminophen at a weight fraction equal to 0.6. Volume fraction of voids were 0.55. The dosage forms were processed at Tₛ=130° C., pₛ=8.2 MPa, and tᵣ=3 s.

FIG. 5D shows compressive force-displacement curves.

FIG. 5E shows tensile strengths derived from compressive force-displacement curves.

FIGS. 6A-6C illustrate schematics of structural configurations of cellular excipients in 2-D. The hexagonal shape of the cells is for illustrative purposes.

FIG. 6A shows a closed-cell structure of an excipient.

FIG. 6B shows a partially open cell structure of an excipient.

FIG. 6C shows an open cell structure of an excipient.

FIGS. 7A-7D illustrate schematics of structural configurations of a cellular thermoplastic excipient (dark gray) and a rapidly eroding excipient (light gray).

FIG. 7A shows a fast the eroding excipient dispersed molecularly or as small particles in the cell walls.

FIG. 7B shows a fast the eroding excipient in the cell walls with a particle size of the order of the wall thickness.

FIG. 7C shows the fast eroding excipient inside the voids.

FIG. 7C shows the fast eroding excipient integrated in the structure. The particle size of the eroding excipient is larger than that of the cells.

FIG. 8 illustrates a schematic of an injection-molding setup to produce cellular dosage forms.

FIGS. 9A and 9B illustrate schematics showing how the final microstructure of the cellular dosage form depends on the injected volume relative to the volume of the mold cavity.

FIG. 10 shows images of cell wall rupturing due to high pressure of gas inside the structure. The cellular dosage form samples were immersed in the dissolution medium.

FIG. 11 shows cellular dosage forms with a volume fraction of voids of 0.6 after immersion in the unstirred dissolution medium. The top images are dosage forms with PEG 20,000. The bottom images show dosage forms with PEO 100,000.

FIG. 12 includes images of dosage forms with PEG 12,000 and volume fraction of voids of 0.55. The top images show exfoliation downwards of a fragment with higher density than water. The time intervals between images were 0.4 seconds. The bottom images show exfoliation upwards of a fragment with lower density than water. The time intervals between images were 0.08 seconds.

FIG. 13 depicts disintegration time of PEG 8000 and PEG 8000-drug composite films. Films were placed in a dissolution medium at 37° C. and the time for the film to break apart was recorded. The calculated effective diffusivity is 4.33×10⁻¹⁰ m²/s for the system with only the PEG 8000 excipient, and 3.67×10⁻¹⁰ m²/s for the excipient-drug system with a drug volume fraction of 0.6. lₚₑₙ is assumed here to be equal to half of the thickness of the film.

FIG. 14 shows sorption tests to determine the amount of water sorbed by the excipient at equilibrium. A dry sample of 10 mg was placed in a dynamic vapor sorption system. The sample was exposed to 95% humidity at 37° C. and the mass of the sample was monitored versus time. From the sample mass at equilibrium and the initial sample mass, the amount of water sorbed can be calculated.

FIG. 15 depicts viscosity of polyethylene glycol solutions versus molecular weight of polymers. The mass of polymer divided by the mass of water added was 0.5. The viscosity of PEO 100k is larger than the viscosity of the lower molecular weight polymers.

FIG. 16 depicts viscosity of polyethylene glycol 12k versus shear rate. The mass of polymer divided by the amount of water was 0.5. If drug is added, mass of drug divided by the mass of polymer is 1.5.

FIGS. 17A and 17B show concentration of the eroding polymer, c₀ of PEG 8k in 0.05 M Phosphate Buffer Solution at pH 5.8.

FIG. 17A shows fraction of drug dissolved versus time at certain angular velocities. The samples were 2.2 mm thick and consisted of 95% excipient and 5% drug by mass.

FIG. 17B shows flux of the eroding polymer versus square-root of rotation rate.

FIG. 18 depicts stress versus engineering strain curves from compression test of melt-processed PEGs and PEO. PEG 1.5k and PEG 8k samples were injection-molded, all others were cast.

FIG. 19A is a semi-log plot of Young's modulus versus molecular weight for selected injection-molded (IM), cast (CM), and cast, strain-hardened (SH) PEGs and PEOs. The data point for injection-molded PEG 8000 was not considered in the statistical analysis.

FIG. 19B is a log-log plot of yield strength versus molecular weight for selected injection-molded (IM), cast (CM), and cast, strain-hardened (SH) PEGs and PEOs.

FIG. 19C is a log-log plot of compressive strength versus molecular weight for selected injection-molded (IM), cast (CM), and cast, strain-hardened (SH) PEGs and PEOs.

FIG. 19D is log-log plot of Strain at fracture versus molecular weight for selected injection-molded (IM), cast (CM), and cast, strain-hardened (SH) PEGs and PEOs.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “agent” refers to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that are man-made in that it is designed, engineered, and/or produced through action of the hand of man and/or are not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized include small molecules, antibodies, antibody fragments, aptamers, siRNAs, shRNAs, DNA/RNA hybrids, antisense oligonucleotides, ribozymes, peptides, peptide mimetics, peptide nucleic acids, small molecules, etc. In some embodiments, an agent is or comprises a polymer. In some embodiments, an agent contains at least one polymeric moiety. In some embodiments, an agent comprises a therapeutic, diagnostic and/or drug.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

As used herein, the term “designed” refers to an agent (i) whose structure is or was selected by the hand of man; (ii) that is produced by a process requiring the hand of man; and/or (iii) that is distinct from natural substances and other known agents.

As used herein, the term “dosage form” refers to physically discrete unit of a therapeutic agent for a subject (e.g., a human patient) to be treated. Each unit contains a predetermined quantity of active material calculated or demonstrated to produce a desired therapeutic effect when administered to a relevant population according to an appropriate dosing regimen. For example, in some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). It will be understood, however, that the total dosage administered to any particular patient will be selected by a medical professional (e.g., a medical doctor) within the scope of sound medical judgment.

As used herein, the term “excipient” refers to a non-therapeutic agent that may be included in a pharmaceutical composition, for example to provide or contribute to a desired consistency or stabilizing effect. Suitable pharmaceutical excipients include, for example, polymers, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

As used herein, the term “substantially”, and grammatical equivalents, refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

### DETAILED DESCRIPTION

It is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Throughout the description, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting.

Described herein are a design, manufacturing, and evaluation of cellular dosage forms capable of releasing drug rapidly. Cell topology and formulation of cellular dosage forms are designed in such a way that the dosage forms exfoliate fragments after immersion in a dissolution medium. A large surface area-to-volume ratio of the exfoliated solid content combined with a soluble, erodible excipient provides rapid drug release. Cellular tablets introduced here satisfy immediate-release requirements and mechanical properties.

Also described herein is a manufacturing process of cellular dosage forms that enable efficient manufacture of them for immediate drug release using inert, non-reactive and non-toxic foaming agents. The process may be efficient because the fluid-based process is substantially predictable and it can be integrated into one single machine with short process time, small footprint, efficient in-process control, reduced capital and operating cost, and short product and process development time. For example, the process includes mixing one or more active pharmaceutical ingredients with one or more excipients, introducing a foaming agent in the melt mixture, dissolving the foaming agent in the mixture so that its concentration in the mixture is homogeneous, introducing a given amount of the mixture into a mold, reducing the pressure of the mixture, and reducing the temperature of the mixture and solidifying the mixture to form a cellular dosage form.

### Microstructure of Cellular Dosage Forms

In some embodiment, cellular dosage forms may comprise multiple gas-filled cells or voids. Cells may be surrounded by a solid which forms a continuous structure comprising one or more pharmaceutically active ingredients and one or more excipients. Cell walls from a solid structure may be removed so that clusters of cells can be formed with interconnected void space. A shape of cells may be convex.

Unlike dense solid or closed-cell matrices, structures with open cells allow rapid percolation of the dissolution medium to the inside of the dosage form. The open cell structure may have the thickness of cell walls as a rate-determining length-scale for drug release instead of the thickness of the dosage form. Open-cell dosage forms with hydrophilic, soluble polymeric excipients exfoliate small fragments when cell walls are penetrated by a dissolution medium and unable to resist the external forces applied on them. A large surface area-to-volume ratio due to the exfoliated fragments and the erosion of the open-cell structure may increase drug release rate by more than an order of magnitude compared with dense solid or closed-cell counterparts. A high solubility of the excipient speeds up erosion of the exfoliated fragments and the dosage form, thus speeding up the dissolution rate of drug from such fragments.

In some embodiments, cell sizes, as well as projected dimensions of walls that are removed from a structure, may be on the micro- or meso-scale. Micro-scale or meso-scale channels in cellular dosage forms can lead to fast fluid flow by capillary forces inside the channels. In some embodiments, cells have average size (e.g., average channel width, and/or average internal diameter) in a range from 3 μm to 1200 μm, from 5 μm to 1000 μm, or from 10 μm to 1000 μm. In some embodiments, cells have average wall thickness, h₀, in a range from 1 μm to 500 μm, from 1 μm to 300 μm, or from 3 μm to 300 μm.

In some embodiments, solid cellular dosage forms may have sufficient mechanical strength to be handled during manufacturing, shipping, and use by end-users. Increasing a volume fraction of voids and decreasing strength and toughness of excipients decreases tensile strength of microstructures. Tensile strength of dosage forms may be higher than 0.05 N/mm² 0.25 N/mm², or 0.3 N/mm². Without wishing to be bound by any particular theory, dissolution rates of dosage forms may be inversely correlated to mechanical strength.

In some embodiments, solid drug contents of an immediate-release solid dosage form may be converted into molecularly dissolved units less than about 30 minutes, 25 minutes, 20 minutes, 15 minutes, 10 minutes, or 5 minutes after ingestion.

### Dissolution of Cellular Dosage Forms

### Percolation:

When at least two sides of gas-filled clusters are opened to a dissolution fluid, the dissolution fluid percolates the clusters rapidly due to capillary forces. Clusters opened to the dissolution fluid on one side only, may not be not percolated rapidly. Air inside the clusters develops a capillary pressure that is in equilibrium with the capillary forces that draw the fluid into the channel.

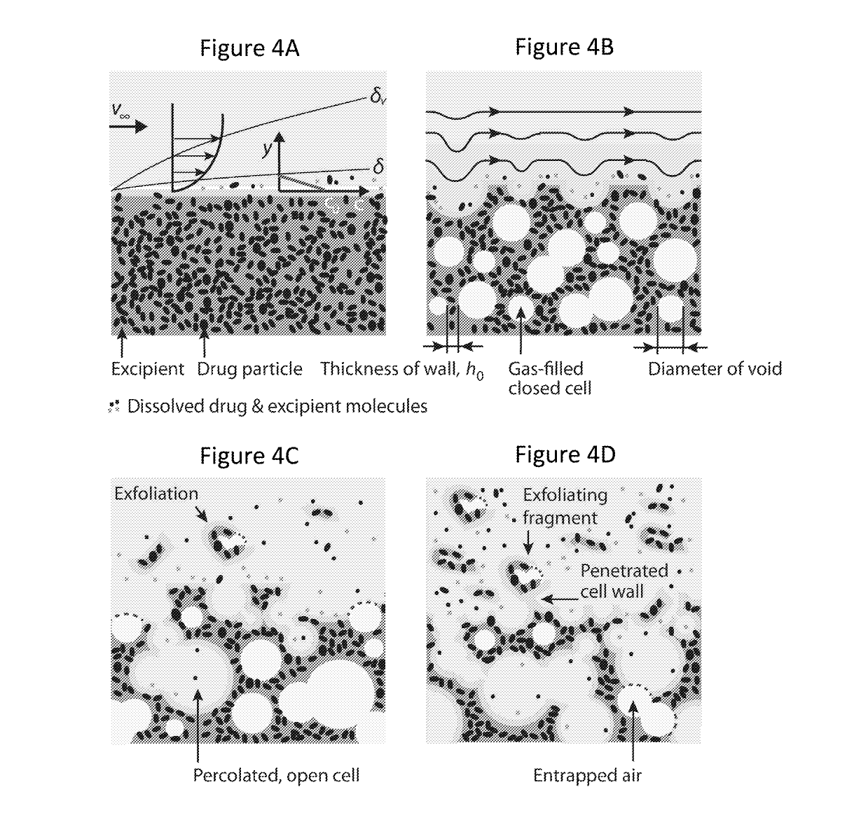

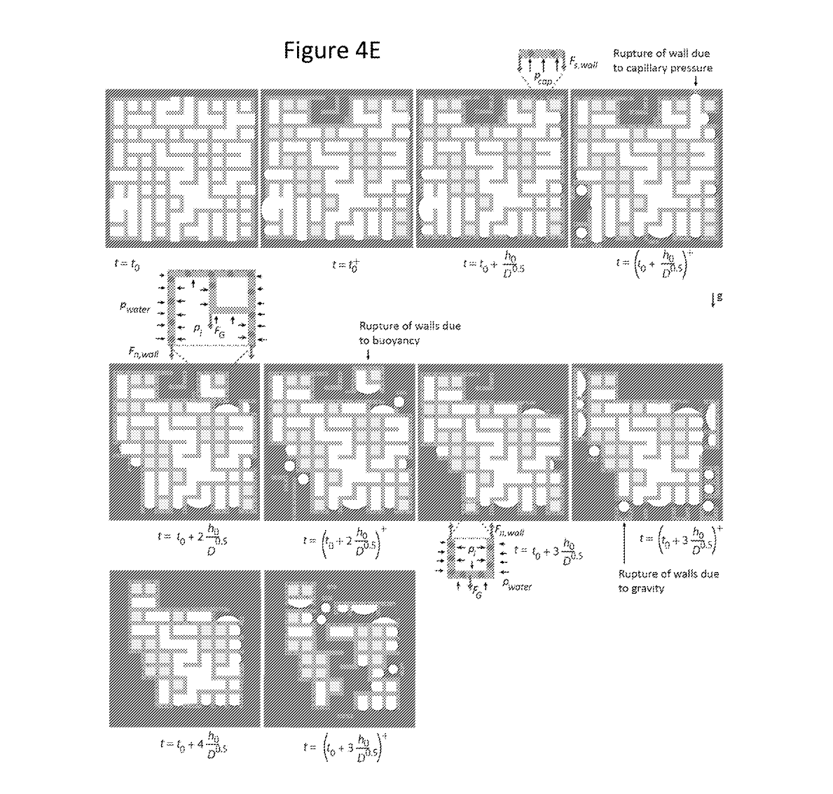

After immersion of a dosage form in a dissolution fluid, the surface of the dosage form may be penetrated by the dissolution medium, and the penetrated walls (e.g., in contact with a pressurized cluster) may be ruptured due to the capillary pressure. This rupture creates an additional opening that exposes the (pressurized) cluster to the dissolution fluid, enabling rapidly percolation of dissolution fluid in the cluster. Subsequent to the percolation, the solid inside the cluster that is in contact with the percolated dissolution fluid is penetrated. It weakens further areas of the structure, and so allows more walls connected to a pressurized cluster to be penetrated and to be ruptured, and so more fluid to be percolated inside the dosage form. An exemplary dissolution process of a cellular dosage form is illustrated in FIG. 4E.

### Exfoliation:

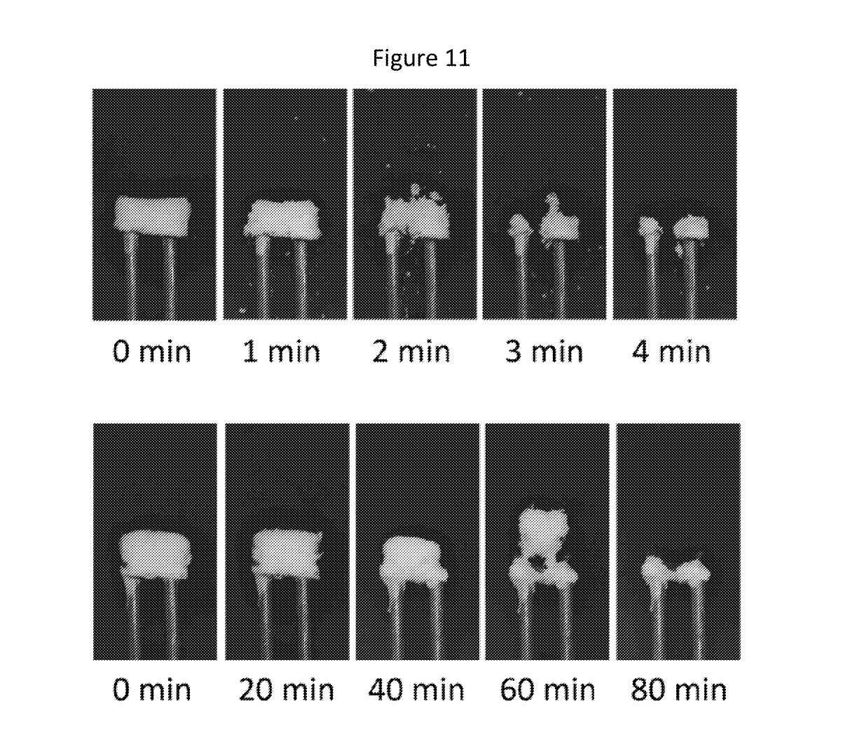

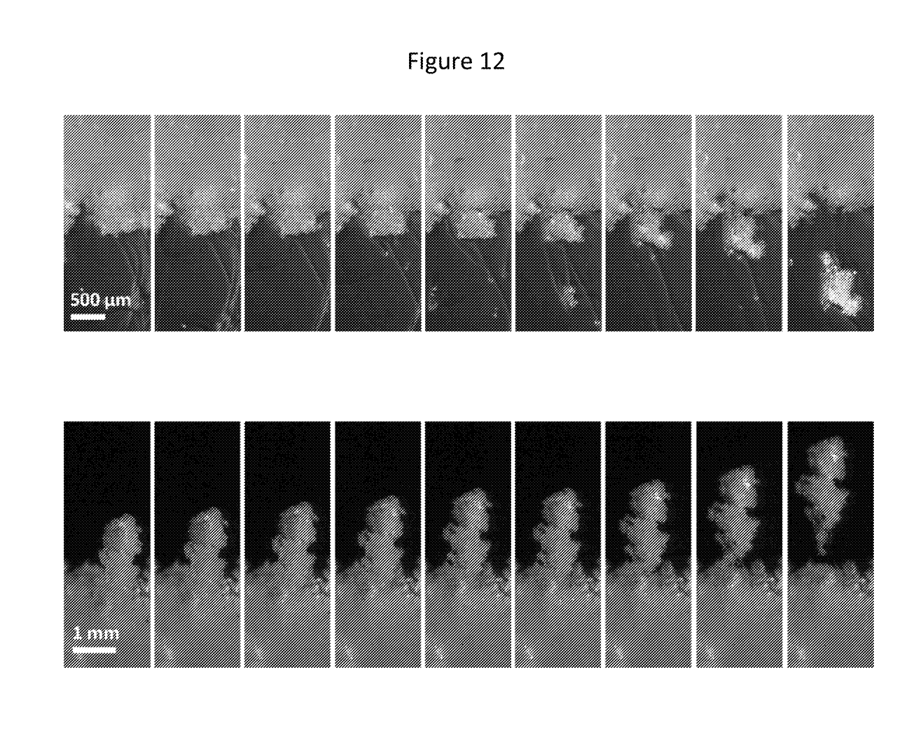

During a dissolution process, a dosage form may have lower mechanical strength than its initial dosage form due to penetration of a dissolution fluid. As mechanical strength of the penetrated formulation is low, applied forces (e.g., gravitational forces, shear forces, or imbalances in hydrostatic pressure) on dosage forms during dissolution may rupture the structure. For example, the above-mentioned forces may cause exfoliation and removal of fragments from the structure, as shown in FIGS. 11 and 12.

A low viscosity of a swollen excipient may results in a high exfoliation rate. Because the penetrated excipient is more fluid-like than solid-like, it can be well characterized by its shear viscosity. A penetrated excipient may have a shear viscosity below 100 Pa·s, 50 Pa·s, or 25 Pa·s. By controlling the viscosity of the penetrated excipient, the exfoliation rate of fragments may be controlled.

### Control Parameters:

An exfoliation rate of fragments may be controlled by a fraction of open cells that are part of a cluster with respect to the total number of cells, an average cell wall thickness distance, and a velocity of a penetrating dissolution fluid that advances into the solid excipient at a solid-liquid interface.

A fraction of open cells may determine how many walls must be penetrated and ruptured in sequence until a structure is percolated. In some embodiments, the fraction is between 0.3 and 1, between 0.35 and 1, or between 0.4 and 1. A fraction of open cells may further determine the amount (i.e., volume) of residual air entrapped in the dosage form during dissolution. A low fraction of open cells may give a large amount of air entrapped, thus impeding exfoliation. In such cases, a significant amount of the drug inside the dosage form may be released from the original structure into the dissolution medium. A large fraction of open cells may result in a low amount of residual air entrapped, thus not imposing an impediment to exfoliation. In these cases, the drug may be mostly released from the exfoliations into the dissolution medium with increased surface area-to-volume ratio.

An average cell wall thickness may determine how deep the dissolution fluid must penetrate to soften a wall. The smaller this distance is, the larger is the rate of exfoliation. In some embodiments, this distance is between about 1 μm and 500 μm, between about 1 μm and 300 μm, or between about 3 μm and 300 μm.

A velocity of a penetrating dissolution fluid that advances into a solid excipient at a solid-liquid interface may determines how fast a fluid penetrates the solid. For example, if Fickian diffusion is dominant, the diffusion can be characterized by a diffusion coefficient of a dissolution fluid in a formulation. This velocity may be larger than the average thickness of the solid wall divided by the maximum dissolution time, e.g., v>h₀/1800 [um/s], v>h₀/300 [um/s], or v>h₀/150 [um/s].

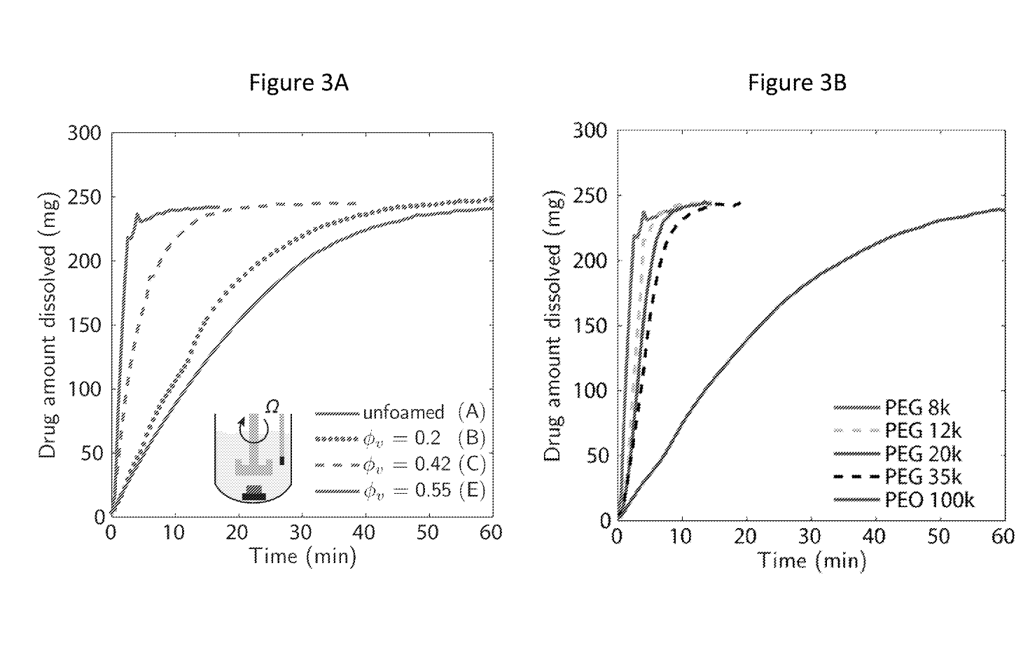

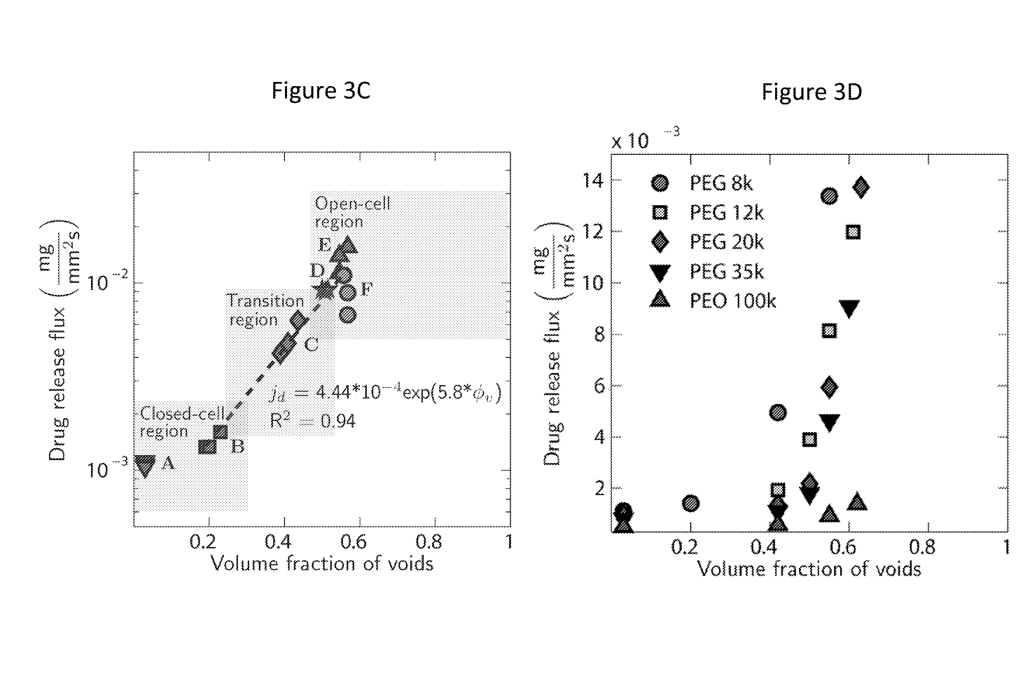

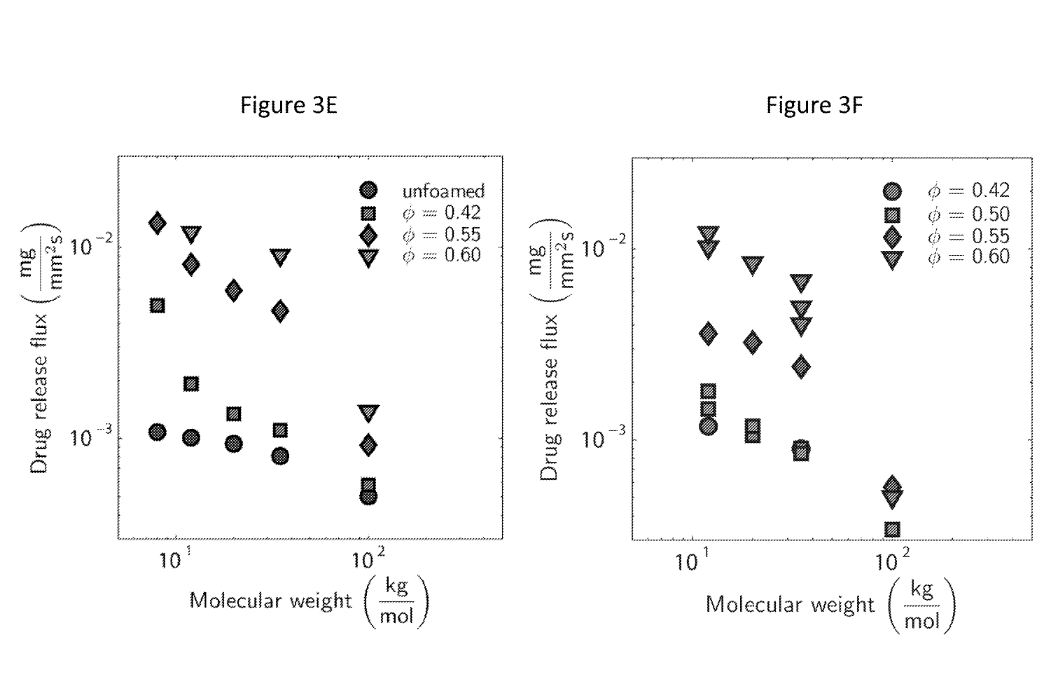

A volume fraction of voids may be related to the three controlling parameters discussed above. As the void volume fraction is increased, the fraction of open cells is increased. Also, the thickness of walls with respect to the cell size is decreased. Therefore, as the volume fraction of voids is increased, the rate of exfoliation is increased. For example, the drug release flux increases exponentially as the volume fraction of voids increases from 0.3 to 0.6, as shown in FIGS. 3C and 3D. Cellular dosage forms may have void volume fraction with respect to total volume, φ_{y}, in a range from 0.2 to 0.85, from 0.3 to 0.8, from 0.35 to 0.75, no less than 0.3, no less than 0.35, or no less than about 0.4.

### Compositions

In some embodiments, excipients may further be soluble in physiological fluids (e.g., water, saline, saliva, and/or gastrointestinal fluid). In some embodiments, excipients may be hydrophilic. A contact angle of a hydrophilic excipient with a dissolution fluid may be less than 90 degree. With those excipients, drugs can be released from exfoliated fragments into the dissolution medium by erosion of the fragments (e.g., erosion of the excipient). Drug molecules also can erode themselves or diffuse through an excipient structure into a dissolution medium, but the diffusion is slower than the rates of drug release that can be achieved by erosion of fragments. Excipients with high solubility in a dissolution fluid provide a means for speeding up the drug release rate.

When excipients are insoluble in and not swellable by physiological fluids, diffusive transport of dissolution medium into the dosage forms and/or drug molecules out of the dosage forms may be rate-determining steps. Dosage forms with excipients insoluble in and not swellable by physiological fluids may not be suitable for immediate drug release, because distances traveled by individual molecules in solution in the time required for immediate drug release are much shorter than the characteristic length-scale of a typical dosage form (several millimeters).

In some embodiments, excipients are selected from the group consisting of polyethylene glycols with a molecular weight above 1,500 g/mol, polyethylene oxides, poloxamers (e.g. poloxamer 188 and poloxamer 407), polymethacrylates (e.g. poly(butyl methacrylate, (2-dimethylaminoethyl) methacrylate, methylmethacrylate) 1:2:1), polyvinylpyrrolidones (e.g. 1-vinyl-2-pyrrolidinone polymer (Povidone) or polyvinylpyrrolidone-vinyl acetate copolymer (Copovidone)), Kollicoat IR, glyceryl behenate, glyceryl distearate, stearic acid, or combinations of these.

In some embodiments, an excipient may have average molecular weight in a range from 1,000 g/mol to 300,000 g/mol, or 2,000 g/mol 200,000 g/mol.

In some embodiments, the melting temperature and/or the glass transition temperature of excipients may be more than 10° C. below the degradation temperature of the active ingredient. Melting temperature and/or glass transition temperature of thermoplastic excipients may be above about 30° C., above about 35° C., above about 40° C., or above about 45° C. The excipient may show a high tendency to solidify when below its melting temperature and/or the glass transition temperature to give a short mold cycle time.

In some embodiments, the viscosity of a polymeric excipient may be too high for injection into a mold and/or the adequate nucleation and growth of microscopic gas bubbles when the excipient is at a temperature just around its glass transition temperature or melting temperature. The viscosity of the polymeric excipient may be reduced for being able to better inject the pharmaceutical material into a mold and/or improved nucleation and growth of microscopic gas bubbles by either increasing process temperature to above the glass-transition temperature or melting temperature of the excipient, or by adding a plasticizer to the thermoplastic polymeric excipient.

In some embodiments, plasticizers may be added to thermoplastic polymeric excipients. Plasticizers may be selected from the group consisting of triethyl citrate, acetyl triethyl citrate, polysorbate 80, and polyethylene glycols (molecular weights<20,000). Similarly, the viscosity of the molten formulation may in some cases be too low for adequate processing. In this case, a high molecular weight polymer or a filler (including but not limited to micro crystalline cellulose, hydroxypropyl methylcellulose, hydroxyethylcellulose, hydroxypropylmethyl cellulose phthalate, cellulose acetate phthalate, noncrystalline cellulose, starch and its derivatives, sodium starch glycolate, and mixtures thereof), may be added to the formulation.

In some embodiments, excipients that resist the penetration of dissolution medium, or the drug from diffusing out, may be eroded away. Drug release rate of dosage forms with erodible excipients may be increased significantly due to elimination of such impediments to drug release.

In some embodiments, rapidly eroding excipients may be added to the formulation that have a characteristic erosion rate (ψ=(solubility×diffusivity^{1/2})/(pi^{1/2}×density) greater than about 1×10⁻⁵ m/s^{/2}, about 2×10⁻⁵ m/s^{/2} or about 5×10⁻⁵ m/s^{/2}. Fast eroding excipients may be selected from the group consisting of sucrose, sorbitol, xylitol, dextrose, maltitol, lactitol, PEGs with a molecular weight of about 4000-70,000 g/mol, mannitol, and isomalt. Furthermore, biocompatible fillers, stabilizers, anti-oxidants, colorants, taste maskers, or other additives commonly used in pharmaceutical formulations may be added to the formulation.

A maximum amount of solid, non-thermoplastic excipients in a system may depend on processability limits. For example, both fast eroding excipients, drugs, fillers and other non-thermoplastic excipients may be in the solid state during processing, whereas thermoplastic excipients are in the plasticized state. About 5-30% of the solid volume may be plasticized to have sufficient fluidity for processing. The maximum cumulative sum of the solid volume fraction of fast eroding excipients and drugs may be about 0.7-0.95. For example, a system that consists of 20% thermoplastic excipient and 30% non-thermoplastic excipient is limited by a solid drug volume fraction of 0.5.

A low volume fraction of excipients is desired for economic reasons (e.g., to save excipient material cost). A high volume fraction may, however, be required if the drug is very potent and the tablet must only contain a few micrograms of it. Below an excipient volume fraction of 0.12, it will become difficult to process the material. For processing reasons, this fraction may be optimally above about 0.2 or 0.25. A lower volume fraction of excipients (e.g., 0.3 to 0.4) is acceptable when the excipient is very soluble and has low molecular weight (e.g. PEG 8000). A higher fraction (e.g., closer to 1) is better for a less soluble excipient and/or an excipient with higher molecular weight.

In some embodiment, cellular dosage forms may include an effervescent agent (e.g., sodium bicarbonate). Volume fraction of the fast eroding excipients with respect to total volume of the dosage form, may be within a range from about 0.01 to about 0.1 Effervescent agents (e.g., release CO₂) may affect gastrointestinal pH.

In some embodiments, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator).

In some embodiments, active ingredient may be in crystalline or amorphous phase or dissolved in the excipient or dispersed in the excipient. In some embodiments, the drug particle size is 100 nm-500 um or 500 nm-500 um. For example, Acetaminophen is chosen as a model drug (e.g. particle size of about 40 μm), and polyethylene glycol with an average molecular weight of 8,000 g/mol (PEG 8000) is chosen as excipient.

### Manufacturing of Dosage Forms

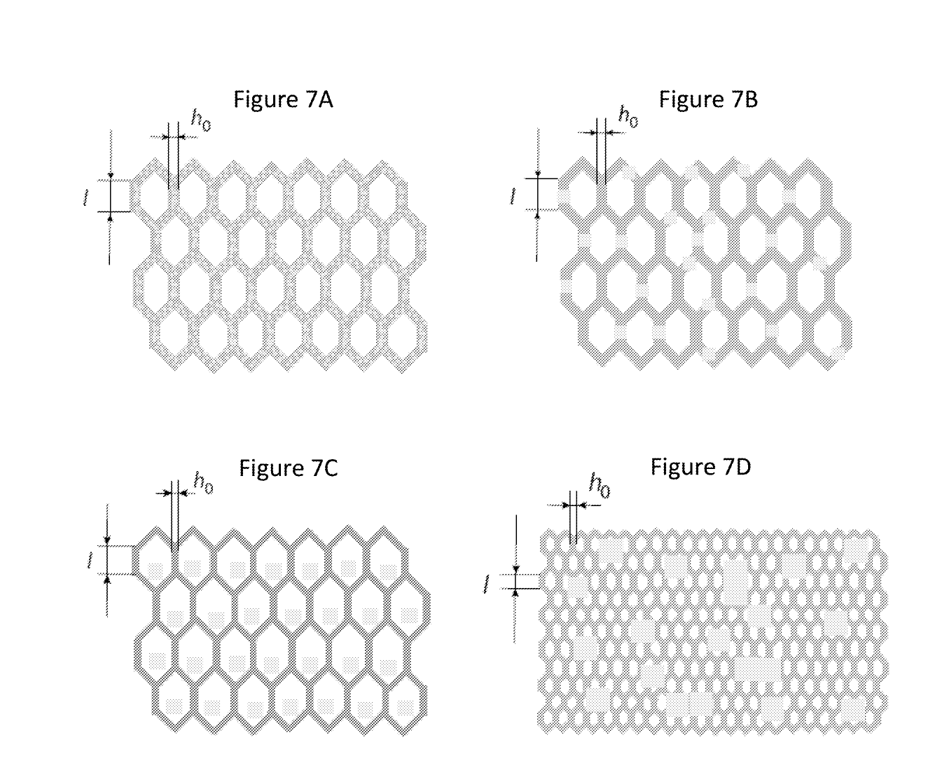

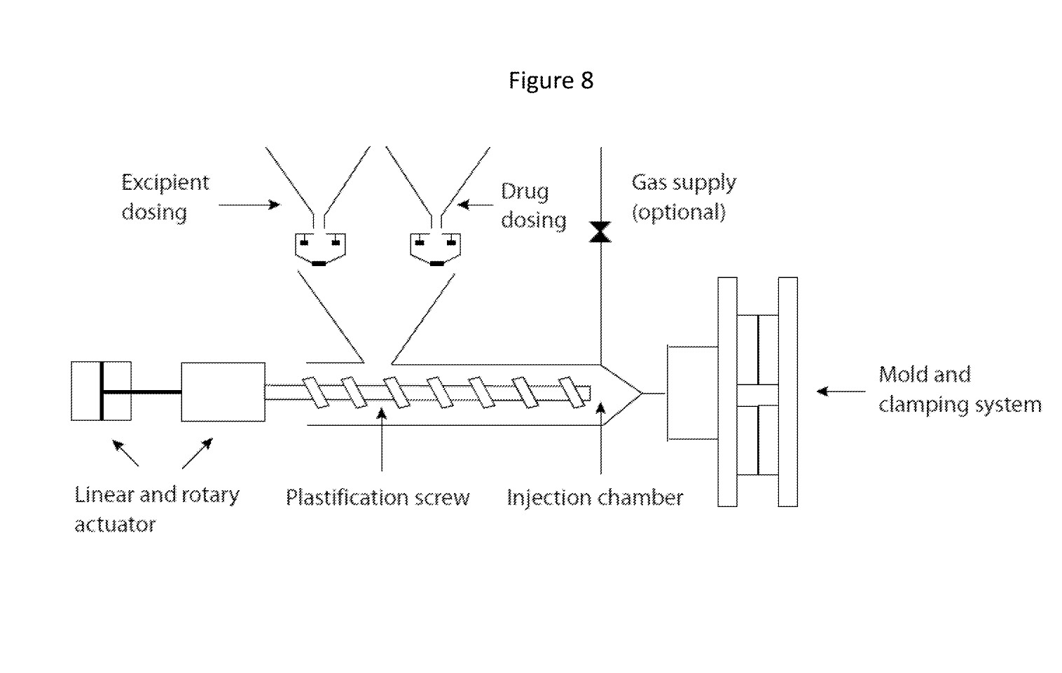

In some embodiments, cellular excipient structures may be manufactured by mixing and injection molding. Dosage forms with high drug volume fraction (e.g., drug volume fraction with respect to solid phases) may require less mixing time and force compared with the manufacture of traditional dosage designs. For example, mixing step may be integrated into the injection molding-machine, as shown in FIG. 7. Drug and excipient may be dosed into the injection-molding machine where the material is mixed, plasticized under heat, and injected into a mold. The machine to manufacture the dosage form is not limited to an injection-molding machine. In some embodiments, it may also comprise, for example, feeders to dispense drug and excipient into an extruder, which can be either single-screw or twin-screw, combined with an adapted, customized setup to mold and shape the dosage form.

In some embodiments, active ingredients and excipients may be mixed in granular forms before a fluidizing process (e.g., melting). In some embodiments, active ingredients and excipients may be mixed after fluidizing either excipients, or both excipients and active ingredients. Excipients may be a solid at a temperature of 20° C. but soften at a temperature of about 30° C. to about 190° C. In certain embodiments, active ingredients and fluidized excipients may be mixed in the presences of shear forces (e.g., extrusion process) at a temperature between about 40° C. and 200° C. At the end of the mixing process, a coefficient of variation of the active ingredient in the mixture may be below 5%. Temperature of the mixture may be homogenized across the mixture during the mixing stage.

In some embodiments, a foaming agent (e.g., a gas and/or a supercritical fluid) may be introduced into a mixture. A foaming agent may be introduced after excipients are fluidized. A foaming agent may be introduced after or before mixing is completed. In certain embodiments, a foaming agent may be introduced by a nozzle to an extruder. A nozzle may have a porous end so that small bubbles of the foaming agent can be formed in the mixture of active ingredient and excipient. The amount of foaming agent added (e.g., concentration, mass, volume) may be adjusted by the pressure of the foaming agent in the nozzle.

In some embodiments, a foaming agent may be dissolved in a mixture of active ingredients and excipients so that concentration of the foaming agent is homogeneous in the mixture. This process may be accelerated by applying shear forces on the mixture. An amount of a foaming agent that can be dissolved in a specific excipient is determined by the temperature and the pressure of the mixture. With a higher pressure, the mixture dissolves a larger amount of the foaming agent. A saturation pressure of the foaming agent in the mixture may be in the range of about 2 MPa to about 30 MPa (e.g., about 3 MPa to about 30 MPa).

A pressurized and plasticized mixture of active ingredients, excipients and a foaming agent may be introduced into a mold (e.g., via mold injection). A certain amount of the pressurized mixture is dispensed into a mold that allows shaping to a final dosage form. The mold can be open or closed. The injected volume of the mixture may be less than the mold capacity. The pressure in the mold cavity may be reduced (e.g., to a pressure lower than the pressure of the material before injection, to atmospheric pressure or to a pressure above atmospheric pressure or below atmospheric pressure). This pressure release may reduce the solubility of the foaming agents in the plasticized pharmaceutical material, initiating nucleation and growth of gas bubbles.

In some embodiments, nucleation may be either heterogeneous or homogeneous (e.g., at the interface between excipient and solid drug or thermoplastic excipient and/or solid additive, inside the excipient phase). For example, heterogeneous nucleation creates gas bubbles at the interface of drug particles and the polymeric excipient, which may result in drug particles fully or partially surrounded by voids as illustrated in FIG. 7C. Some particles may be partially surrounded by voids as shown in FIGS. 7B, and 7D, or the particles may be inside the wall as shown in FIG. 7A. Homogeneous nucleation may create cells just within the thermoplastic excipient, promoting active ingredient particles and other particles to be surrounded by thermoplastic excipient, as shown in FIGS. 7A, 7B and 7D.

In some embodiments, nucleation types may be controlled by manipulating interfacial energy of liquid thermoplastic excipients and solid drug phases. High interfacial energy lowers nucleation activation energy, promoting heterogeneous nucleation. Low interfacial energies (e.g., the plasticized phase/solid phase interface, the polymer/bubble interface) result in more homogeneous nucleation. A higher degree of supersaturation (e.g., concentration of dissolved gas or supercritical fluid in the mixture at the given temperature and pressure minus solubility of gas or supercritical fluid in the mixture at the given temperature and pressure) of the dissolved gas or supercritical fluid is required in this case to achieve high nucleation rates. In heterogeneous nucleation, high nucleation rates can be achieved even at lower degrees of supersaturation. In heterogeneous nucleation, therefore, the partial pressure of gas or supercritical fluid can be reduced to achieve a given nucleation rate.

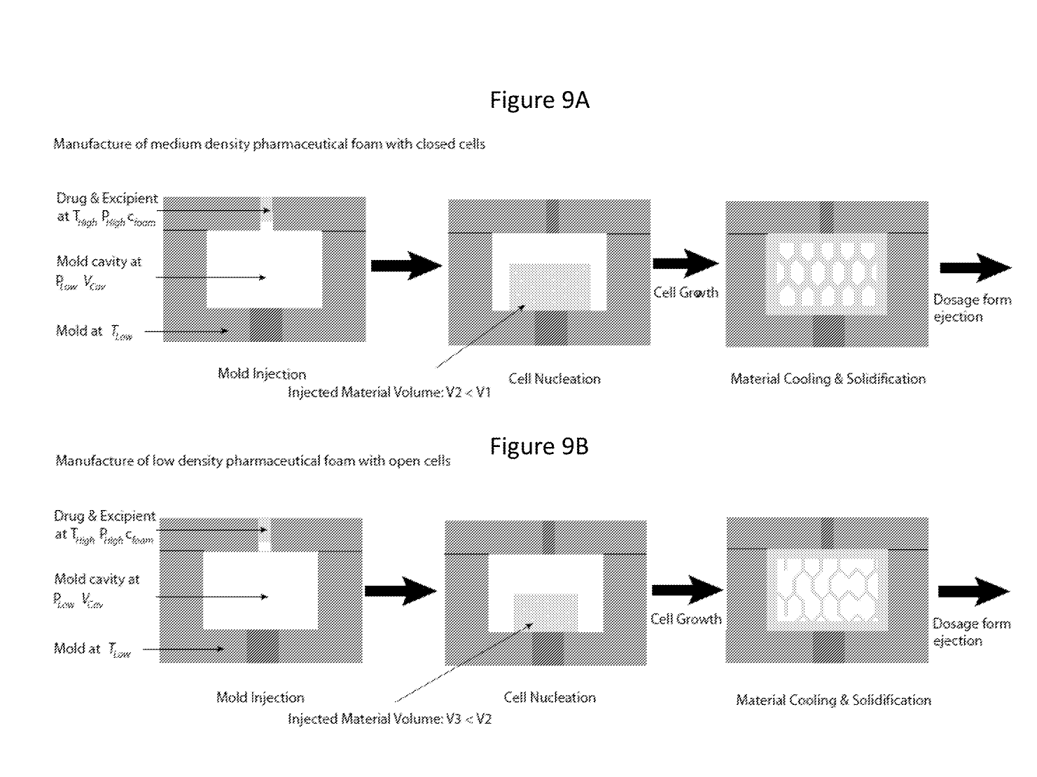

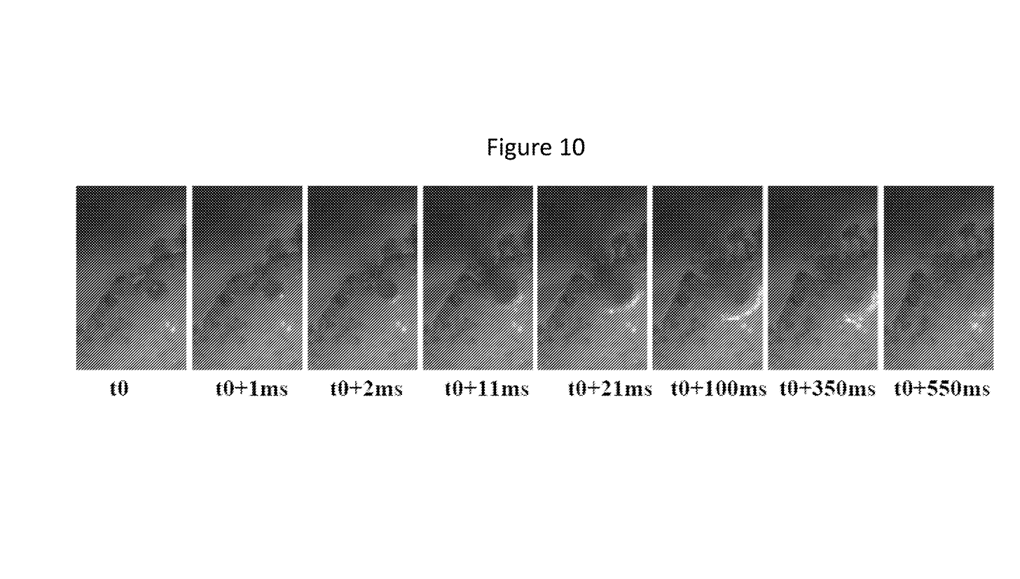

Growth rates of gas bubbles in a mold cavity may be related to gas concentration and gas solubility (e.g., degree of supersaturation of the foaming agent in the pharmaceutical material), pressure of gas in the cell bubbles (e.g., determined by the bubble size, the surface energy between gas and thermoplastic excipient and the external pressure applied on the pharmaceutical material i.e., the pressure inside the mold cavity), diffusion coefficient of gas in the polymer, and viscosity of the pharmaceutical material. Cell growth may be controlled by controlling the temperature-time profile and the pressure-time profile of the pharmaceutical material and the foaming agent. The injected volume of pharmaceutical material, V, relative to a volume of a mold cavity, V_{cav} (FIG. 9A-9C) may determine a void volume fraction of cellular dosage forms, as well as a morphology and characteristics of the cellular structure as shown in FIGS. 9A and 9B. When V/V_{cav} is small, the resulting void volume fraction is large. A diameter of a cell relative to a thickness of a cell wall is large. When V/V_{cav} is small, open cells may be formed. Open cells are resulted from rupture of cell walls between voids. For example, a cell wall may break due to high pressure differential between two cells. A cell wall between two growing cells may be opened up as soon as the two growing cells touch each other.

When V/V_{cav} is large, a diameter of a cell relative to a thickness of the cell wall is small and the cell wall may not be ruptured. Thus, closed cells may be formed wiₜₕ high V/V_{cav}. This process allows the production of a large range of cell topologies.

In some embodiments, the mold may be open after injection of the pharmaceutical material. In this case, the expansion of the material is not constrained and the resulting cell topology may be determined by the temperature-time profile and the pressure-time profile applied on the pharmaceutical material and the foaming agent. A mold to shape the surface that was initially open may be applied before the cellular dosage form is fully solidified. Injection temperature, mold temperature, and dosage form geometry must be adjusted, not only to maximize process rate, minimize the amount and unit cost of materials used, minimize capital cost and operating cost, or minimize the amount of material wasted, but also to obtain the desired microstructure and physiological properties, preferably with at least partially open cells. In some embodiments, a multi-phase dosage form may be a coated dosage form or a dosage form consisting of multiple phases, each phase comprising one or more active pharmaceutical ingredients. An example of a technology to produce multi-phase molded products is over-molding. Dosage form molding by over-molding can be implemented in a continuous process using a variety of mold technologies, for example, core pullback, rotary molding, or rotary cube molding technologies. A coated dosage form may also be produced by co-injection molding. In co-injection molding, coating and core materials are injected into a same mold cavity so that the coating material forms a skin to cover the core. Typically, the coating is first injected into the mold cavity. As soon as the coating material touches the cold surface of the mold, it solidifies and forms a surface skin. Coating materials must have the desired thermoplastic properties and required functional properties (e.g., dissolution time, moisture barrier, appearance, color, taste, etc.). The core may be injected subsequently, on top of the coating covering the surface.

### Theoretical Explanation of Dissolution

Dissolution of polymers starts with penetration of dissolution medium into the solid matrix followed by disentanglement of the polymeric chains. When the eroding surface is exposed to the free-flowing dissolution medium (e.g. the Peclet Number Pe>>1), the disentangled polymer molecules are then transported by convection from the eroding surface through a thin concentration boundary layer into the dissolution medium (FIG. 4A).

Flux of the removed polymer may be considered as the steady-state convective mass transfer from the flat surface into a dilute Newtonian viscous fluid. Dependences of viscosity and diffusivity on polymer concentration in boundary layers are neglected. The flux of the eroding polymer can be expressed as:

$$j = const \frac{{Dc}_{0}}{{D}_{0}} {Re}^{\frac{1}{2}} {Sc}^{\frac{1}{3}} \qquad ( 1 a )$$

where Re is the Reynolds number, Sc is the Schmidt number, const is a geometry-dependent constant, D is a diffusivity of excipient in a dissolution medium, D₀ is a length of plate or disk (e.g. where the excipient erodes from), and c₀ is a concentration at the solid-liquid interface of the eroding polymeric matrix.

Erosion time, τₑᵣ, of the solid polymeric disk or flat plate eroding from one side can be:

$${τ}_{er} = \frac{{ρ}_{s}}{j} {H}_{0} \qquad ( 1 b )$$

where ρₛ is the density of the plate or disk, and H₀ is the thickness of the plate or disk.

For example, the erosion time was estimated as 28 minutes for the 2.5 mm thick sample. The calculated value is lower than the experimental result with t_{0.8}=28.54 minutes (see Table 1).

Referring to FIG. 4B, the incorporation of closed cells into a solid matrix increases the eroding surface area (A) compared with the area of the non-porous structure (A₀). The flux of the eroding excipient in FIG. 4B is expected to be higher than FIG. 4A. When exposed cells are assumed to be hemi-spherical, then A=A₀ (1+φ^{v}). Assuming that the streamlines follow the surface dimples and there is no turbulence due to the surface rough-ness, the amount of eroding polymer from a closed cell structure is increased roughly in proportion to the increase in surface area (i.e., j=Aj₀/A₀=(0+φᵥ)j₀). According to this model, the dissolution time decreases by a factor of about 1.2 if the volume fraction of voids is increased from the unfoamed form to 0.2, which is fairly close to the experimentally observed factor of 28.54/21.8=1.31 as shown in Example 3.

Gas-filled open-cell structures develop a pressure difference across the air-liquid interface due to capillarity, when surrounded by dissolution medium. The pressure difference is inversely proportional to the radius of the channel. The dissolution medium percolates through the smaller pores into the dosage form, and the air in turn escapes through the larger channels. Viscous effects limit the speed of percolation. The percolation time, τ_{perc} is as follow:

$${τ}_{perc} = \frac{2 {l}_{perc}^{2} {μ}_{f}}{γ r cos θ} \qquad ( 2 )$$

where l_{pew} is the percolation length, r the radius of the capillary conduits, y the surface tension of the dissolution medium, and θ the contact angle.

Due to the non-uniform ratio of capillary forces to viscous forces in the heterogeneously sized channels, displacement of air by an immiscible phase is dominated by fingers that promote the formation of air clusters entrapped inside the structure. Such clusters are stable if they develop interfaces where the surface forces are in equilibrium with buoyancy and viscous forces. Stable interfaces between air and the percolating liquid develop across channels of equal size in the open-cell dosage forms.

Dissolution medium penetrates excipients in a drug-excipient skeleton. Penetration of solvent into excipients can be adequately described by a concentration-dependent form of Fick's law when the rate of solvent diffusion into the polymer is much less than that of polymer relaxation (i.e., the polymer chains quickly adjust to the presence of the penetrant and hence do not cause diffusion anomalies). This typically is the case if the polymer has a comparably small molecular weight and is in the rubbery state, well above its glass transition temperature (as in the present system). Therefore, the time for penetration of a cell wall can be approximated by:

$${τ}_{pen} = \frac{{l}_{pen}^{2}}{{D}_{eff}} \qquad ( 3 )$$

where lₚₑₙ is the penetration length and D_{eff} is the effective diffusivity, which is about 3.67×10⁻¹⁰ m²/s in the present system. Hence, in some embodiments, τₚₑₙ=23 seconds if lₚₑₙ=100 μm (of the order of the thickness of walls of the dosage forms).

Penetrated cell walls have reduced mechanical strength, and break up into small fragments (exfoliations) as soon as the walls that hold the structure together cannot resist such forces as the hydrostatic forces due to entrapped air, the exfoliating fragment's own weight, or the shear stress exerted by the surrounding fluid. It was assumed that a fragment exfoliates if the wall with the greatest lₚₑₙ that that connects the fragment to the structure is penetrated (FIG. 4D). (lₚₑₙ=h₀/2, for a cell wall thickness of h₀, if the dissolution medium penetrates from both sides, and lₚₑₙ=h₀ if the dissolution medium is on one side and entrapped air on the other.) It may be further noted that as long as the structure is undivided, the rate of erosion of cell walls is much smaller than that of penetration (erosion without convection applies as the fluid is standing in the percolated channels). Exfoliations, however, are exposed to the free-flowing dissolution medium and thus eroded by convection. By Eq. (1b), an exfoliation with a nominal size equal to three times the thickness of the wall (a typical size observed in the experiment) is eroded in about 51 seconds if h₀=100 μm. Therefore, summing up τ_{perc}, τ_{pen,ex} (the time to exfoliate the structure), and the time to dissolve an exfoliation, a dissolution time of about 1-4 minutes is obtained for an open-cell microstructure with h₀ between 100 μm and 200 μm. These values are in close agreement with the experimental results obtained.

### EXPERIMENTAL EXAMPLES

### Example 1

### Preparation of Cellular Dosage Forms

This example demonstrates an exemplary fabrication of cellular dosage forms. Acetaminophen and polyethylene glycol 8,000 were selected as the active ingredient and the excipient for this example.

### Preparation of Cellular Dosage Forms:

Acetaminophen powder was first sieved using a stainless steel mesh with a nominal opening of 53 μm (size No. 270). The drug particles were then combined with solid polyethylene glycol 8,000 (PEG 8000) flakes to give a formulation of 63% Acetaminophen and 37% PEG 8000 by weight. The mixture was then heated to 90° C. and kneaded until a uniform paste was formed. Subsequently, an aliquot of the paste was put in a stainless steel mold held at 25° C. The aliquot was compressed and cooled to give a cast disk with diameter 13 mm and thickness 2.5 mm. The disk was used as reference of the unfoamed samples. For preparation of the cellular dosage forms, the disk was placed in a sample holder with an inside diameter of 13 mm. The sample was then soaked in a pressurizable oven for 50 minutes at certain temperatures and pressures. The gas used in the oven was nitrogen and the pressure was applied using a Supercritical Fluid System (Trexel, Inc.). Subsequently, the pressure was released in a time τᵣ. The oven was then opened and the temperature of the disk reduced to room temperature using an industrial fan. The cooling time (time to cool the sample to about 35° C.-45° C.) was about 1 minute.

Cellular dosage forms were fabricated by first soaking in nitrogen a paste composed of uniformly distributed solid drug particles at a volume fraction of 0.6 in molten excipient. The soaking temperature, Tₛ, was between 70° C. and 130° C., well above the melting temperature of the excipient but below the melting temperature of the drug. The soaking pressure, pₛ, was 4.1-6.9 MPa. After the system was equilibrated, the pressure was gradually released to atmospheric in a time τᵣ, which was either three seconds or one minute. Then the sample was solidified by cooling to room temperature.

### Example 2

### Images and Characteristics of Microstructures

This example demonstrates exemplary characterizations of microstructures in cellular dosage forms using Scanning Electron Microscope images.

### Scanning Electron Micrography (SEM):

A cross sectional surface of the dosage form that shows its microstructure for SEM imaging was obtained by first scoring the sample with a razor blade and then breaking it along the score. A Zeiss Merlin High Resolution SEM with a GEMINI column was used to take the images. Imaging was performed with an in-lens secondary electron detector. An accelerating voltage of 5 kV and a probe current of 95 pA were applied.

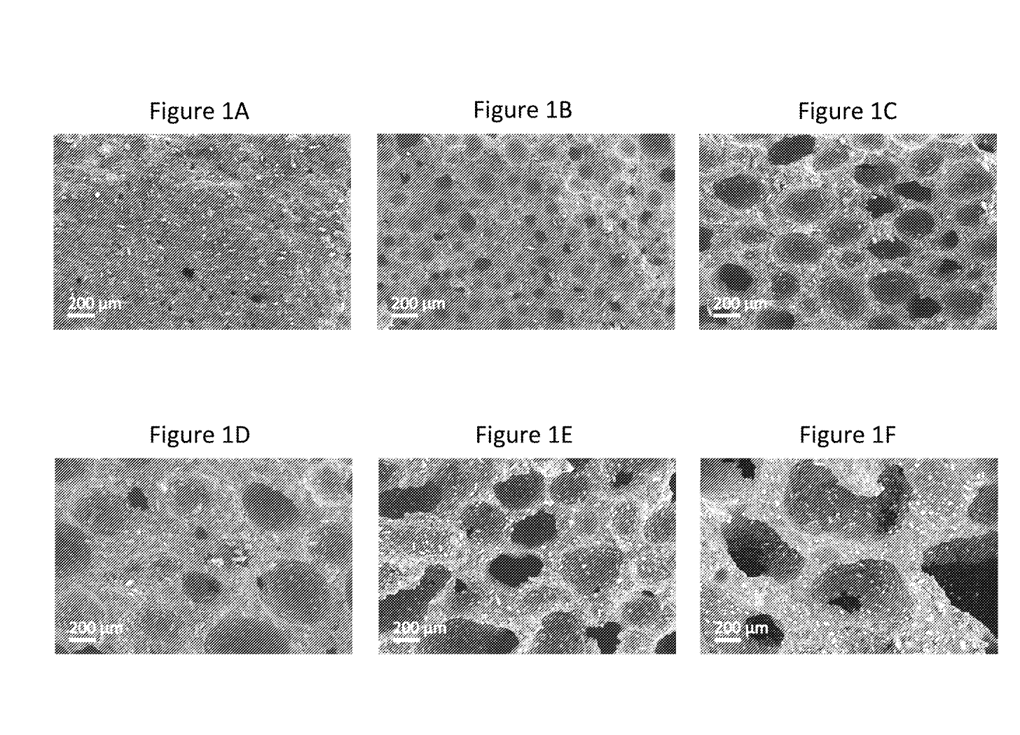

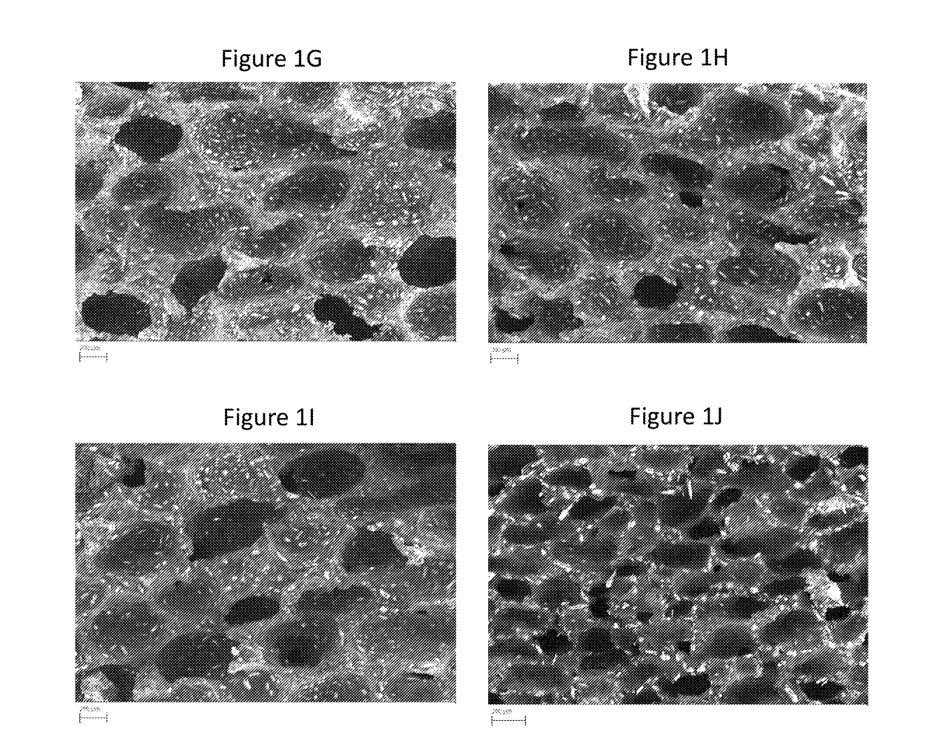

Referring to FIG. 1, it shows morphologies of structures may be tailored by adjusting the process conditions. High Tₛ and pₛ increased the void volume fraction and the fraction of open cells. τᵣ only minimally affected the void volume fraction, but had a large effect on the diameter of voids and further affected the resulting fraction of open cells. By controlling the temperature-time and the pressure-time profile, a variety of tailored structures could be produced, including topologies with clusters of interconnected cells in the void space (open cells). The cellular dosage forms just consist of a polymeric excipient and the drug substance, whereas the foaming agent used is inert and does not leave any residues behind that could potentially be toxic or impair the stability of the dosage form.

The structure and properties of the cellular forms were compared with a cast specimen with the same formulation (unfoamed structure). All the dosage forms tested were 13 mm dia. disks with thickness proportional to the volume fraction of the voids, φ_{y} (H₀=2.5 mm if φᵥ=0).

### Determination of the Fraction of Open Cells, Cell Size, Thickness of the Solid Wall:

The individual cells were observed with the SEM images. The ruptured cell walls (e.g., two cells are connected) were identified. The fraction of cells connected to at least one other cell was determined with respect to the total number of cells seen on the image. Alternatively, more precise determination of the fraction of open cells may be possible from micro CT images or from nano CT images that do not require destruction of the sample.

### Determination of Cell Size:

The individual cells were observed with the SEM images. The maximum and minimum dimensions of each cell were averaged.

### Determination of the Thickness of the Solid Wall:

The individual cells were identified on the SEM images. The cell walls between a cell and its neighboring cells were identified. The average thickness of each of these walls was determined from the SEM images. The average thicknesses of each of these walls were averaged over all the walls. The standard deviation of the average thicknesses of each of these walls was calculated. Alternatively, more precise determination of the wall thickness may be possible from micro CT images or from nano CT images.

Comparison of the Structure with a Random Structure:

The SEM images were compared with random structures generated by the computer. In our structure, the cells are distributed more evenly and thus less clustering of void space and solid space is observed compared with a random structure. The cells are therefore arranged in the dosage form in a means more ordered than random.

### Determination of the Volume Fraction of Voids:

The volume fraction of voids was determined by dividing the difference in volume between the foamed dosage form and the unfoamed dosage form with the volume of the foamed dosage form.

### Example 3A

### Dissolution of Cellular Dosage Forms

This example demonstrates exemplary dissolution tests of cellular dosage forms showing that the dosage forms are suitable for immediate drug release.

### Dissolution Testing:

The dosage form was first attached to a ring disk. The sample was then placed at the bottom of a dissolution vessel (within a Sotax dissolution bath) which was filled with 900 ml of 0.05 M phosphate buffer solution (using sodium phosphate monobasic and sodium phosphate dibasic) at pH of 5.8 and the temperature of 37° C. The solution was stirred using a paddle rotating at 50 rpm. The concentration of dissolved drug was measured by UV absorption at 244 nm using a fiber optic probe with a path length of 2 mm (Pion, Inc.).

### Determination of the Dissolution Time, t_{0.8}:

The time to dissolve 80 percent of the drug content was determined from the curves that show the drug amount dissolved versus time.

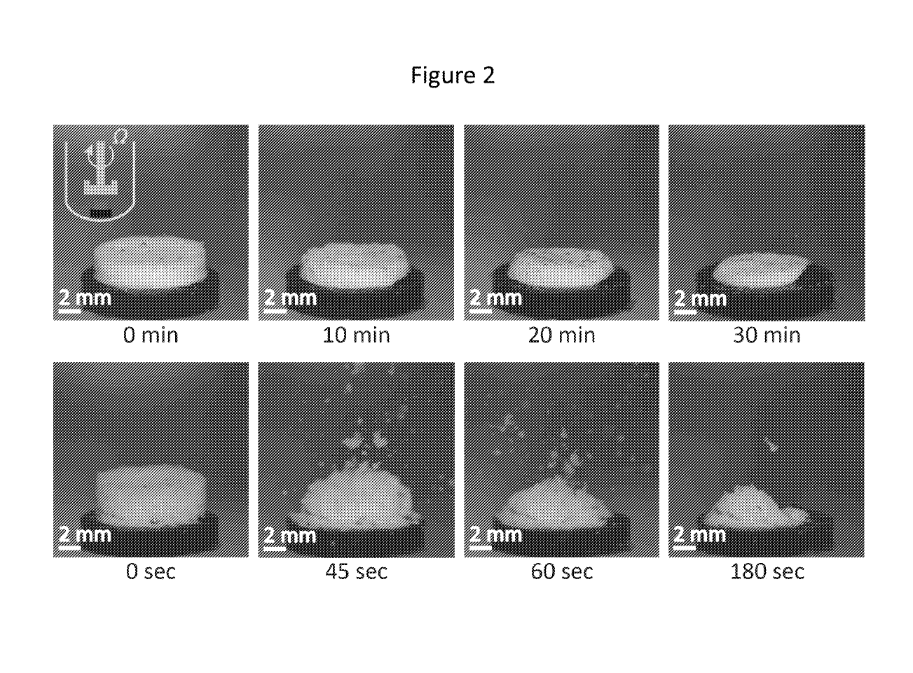

Snapshots of dissolving closed-cell and open-cell dosage forms are shown in FIG. 2. The dosage forms were attached to a ring at the bottom of a dissolution vessel and the medium was stirred by paddles rotating at 50 rpm. Soon after the open-cell dosage form was immersed in the dissolution medium, 0.05-2 mm thick exfoliations were released. The exfoliations then rapidly dissolved, many of them in a few seconds. The closed-cell form eroded by a continuous decrease in size, without releasing visible exfoliations.

FIG. 3A shows the amount of drug dissolved versus time of selected dosage forms. The slopes of the curves decrease with time (primarily because of decreasing surface area) until the curves reach a plateau. The time taken to dissolve 80% of the drug content of the dosage form, t_{0.8}, a commonly used measure for the dissolution time of immediate-release solid forms, is extracted from these curves. The results obtained along with the respective microstructural parameters of the dosage forms are listed in Table 1. Most strikingly, cellular dosage forms, with φ_{y}=0.55, the fraction of open cells equal to 0.69, a wall thickness of 58 μm, and a diameter of the voids of 321 μm, allow to reduce t_{0.8} from about 29 minutes (dense solid-matrix) to only two minutes.

Table 1A below illustrates microstructural, mechanical and dissolution properties of the cellular dosage forms.

TABLE 1A

| | Microstructural Parameters | | | | Properties | |
|---|---|---|---|---|---|---|
| | Volume | | | Fraction | | Maximum |
| | fraction | Diameter | Thickness | of | | com- |
| | of | of voids | of walls | open | t_{0.8} | pressive |
| Process | voids | (μm)* | (μm)* | cells | (min) | force (N) |
| A | 0.03 | 48 ± 25 | — | 0 | 28.54 | 121.2 |
| B | 0.2 | 141 ± 31 | 78 ± 33 | 0.1 | 21.8 | 76.25 |
| C | 0.42 | 253 ± 59 | 76 ± 42 | 0.32 | 6.21 | 54.88 |
| D | 0.49 | 380 ± 79 | 111 ± 54 | 0.57 | 3.37 | 28.81 |
| E | 0.55 | 321 ± 63 | 58 ± 33 | 0.69 | 2.3 | 20.81 |
| F | 0.57 | 552 ± 151 | 154 ± 69 | 0.9 | 3.61 | 13.91 |

*mean ± standard deviation (the data are derived from the images shown in FIG. 1A-1D)
The numbers for t_{0.8} and the maximum compression force represent the mean of three samples, whereas the numbers for the volume fraction of voids are the mean of six samples.
Composition of the dosage form: 60% API (Acetaminophen) + 40% excipient (PEG 8000). Amount of API in the sample: 245 mg. Nominal dimensions of disk specimens: diameter = 13 mm and thickness proportional to the void volume fraction (H₀ = 2.5 mm if φᵥ = 0). t_{0.8} is the time taken to release 80% (196 mg) of the drug present in the dosage form.

Average drug release flux versus cell volume fraction is illustrated in FIG. 3B. The flux was calculated by dividing 80% of the drug content with t_{0.8} shown in Table 1 and the projected surface area of the dosage form. The data are categorized into closed-cell region, transition region, and the open-cell region. In the closed-cell region, the drug release flux is increased in proportion to the increase in the volume fraction of voids. As the volume fraction of voids approaches the percolation threshold, which for a random, infinitely large system of overlapping spheres is at φ_{y}˜0.3, clusters of interconnected cells develop. This enables the dissolution medium to percolate part of the void volume, but clusters that block complete passage of the fluid still exist, as shown in FIG. 4C. Several cell walls need to disintegrate sequentially before fragments are exfoliated from the dosage form. As the volume fraction of voids is increased, the smaller, finite clusters are absorbed by the cluster that spans the entire dosage form, promoting rapid disintegration of the structure. The length scale that governs drug release is changed from the size of the dosage form (non-porous or closed-cell forms) to the thickness of a cell wall (percolated open-cell structures). The results obtained for the drug release flux, shown in FIG. 3B, and the dissolution time and the fraction of open cells, listed in Table 1, suggest that the structures comprise mostly open cells if the volume fraction of voids is above about 0.55.

Cast dosage forms as shown in FIG. 4A may not meet the requirement of immediate drug release. Erosion times of the order of 10 minutes, or even less, could be achieved only if such highly soluble, small-molecule excipients as sucrose or sorbitol are used. However, because of the spatio-temporal variances in the gastrointestinal fluid flow, it is not realistic to rely on drug release by convective mass transfer. Moreover, several of these highly soluble small-molecule excipients invade biological tissues and are absorbed by the blood stream to have an adverse biological effect. In addition to that, such molecules are typically very hygroscopic and tend to impair the stability of the dosage form. An alternative is the use of effervescent agents, such as sodium carbonate or sodium bicarbonate, which are typically converted to a salt and CO₂ immediately after contact with gastric fluids, thus enabling rapid release of the drug. Excipients that release CO₂, however, tend to affect gastrointestinal pH, and effervescent agents further tend to have a negative effect on the stability of the dosage form due to their hygroscopicity and reactivity. Optimally, therefore, the dosage form must be designed with chemically inert and biologically inactive polymeric materials as excipients, but non-porous material structures consisting of polymeric materials erode too slowly for immediate drug release.

### Example 3B

### Dissolution of Cellular Dosage Forms

This example demonstrates exemplary dissolution tests of cellular dosage forms showing that the dosage forms are suitable for immediate drug release.

### Dissolution Testing:

Instead of attaching the samples to a disk, the samples were just placed into the dissolution vessel, without attaching anything to them. They were floating in the vessel. All the other aspects of the method were done as described in Example 3A. This method may resemble a dosage form dissolving in the gastrointestinal system more realistically where the dosage form is also not attached to a weight and thus may be floating.

The time to dissolve 80 percent of the drug content of selected dosage forms is given in Table 1B. FIG. 3F shows the drug release flux versus the molecular weight of the excipient. The flux was calculated by dividing 80% of the drug content with t_{0.8} in Table 1B and the projected surface area of the dosage form. This data is compared with the drug release flux obtained by testing the dosage form dissolution properties according to the method described in Example 3A (illustrated in FIG. 3E with data for t_{0.8} shown in Table 2). It is found that the drug release flux is considerably larger if the dosage form is tested according to the method in Example 3A if the volume fraction of voids is 0.42 and 0.55. This is because of differences in the exfoliation rates. The dosage forms tested by the dissolution method shown in Example 3B have lower exfoliation rates at these cell topologies than the dosage forms tested by the dissolution method shown in Example 3A. Thus they rely more heavily on drug release by erosion of the dosage form (i.e., erosion of the excipient). If the fraction of open cells is increased, however, such as at a volume fraction of voids with respect to the total volume of the dosage form equal to 0.6, both fluxes are roughly the same. Thus a larger fraction of open cells and a larger volume fraction of voids with respect to the total volume of the dosage form is required for the dosage form to achieve rapid drug release using the method shown in Example 3B compared with the method presented in Example 3A.

Table 1B below illustrates process parameters and dissolution properties of the cellular dosage forms tested by the method of Example 3B. The dosage form is floating in the medium and not attached to a weight.

TABLE 1B

| | Microstructural and Process Parameters | | | | |
|---|---|---|---|---|---|
| | Volume | | | | |
| | fraction of | | | | |
| Excipient | voids | T_s | p_s | t_r | t_{0.8} (min) |
| Peg 12k | 0.42 | 110° C. | 5.5 MPa | 3 s | 26.13 |
| Peg 12k | 0.5 | 110° C. | 8.3 MPa | 3 s | 17.12 |
| Peg 12k | 0.55 | 130° C. | 8.3 MPa | 3 s | 8.54 |
| Peg 12k | 0.6 | 130° C. | 8.3 MPa | 40 s | 3 |
| Peg 20k | 0.5 | 110° C. | 8.3 MPa | 3 s | 26.1 |
| Peg 20k | 0.55 | 130° C. | 8.3 MPa | 3 s | 9.5 |
| Peg 20k | 0.6 | 130° C. | 8.3 MPa | 40 s | 3.65 |
| Peg 35k | 0.5 | 110° C. | 8.3 MPa | 3 s | 36.1 |
| Peg 35k | 0.55 | 130° C. | 8.3 MPa | 3 s | 12.75 |
| Peg 35k | 0.6 | 130° C. | 8.3 MPa | 40 s | 6.21 |
| Peo 100k | 0.5 | 110° C. | 8.3 MPa | 3 s | 90.46 |
| Peo 100k | 0.55 | 130° C. | 8.3 MPa | 3 s | 54.24 |
| Peo 100k | 0.6 | 130° C. | 8.3 MPa | 40 s | — |

Composition of the dosage form: 60% API (Acetaminophen) + 40% excipient. Amount of API in the sample: 245 mg. Nominal dimensions of disk specimens: diameter = 13 mm and thickness proportional to the void volume fraction (H₀ = 2.5 mm if φᵥ = 0). t_{0.8} is the time taken to release 80% (196 mg) of the drug present in the dosage form.

### Example 4

### Mechanical Characterization of Cellular Dosage Forms

This example demonstrates exemplary mechanical properties of cellular dosage forms, showing that the dosage forms are mechanically stable.

### Mechanical Testing:

Diametral compression tests were conducted using a Zwick Roell mechanical testing machine equipped with a 2.5 kN load cell and compression platens. The relative velocity of the platens was 1 mm/min. The test was stopped as soon as the specimen fractured, or the load dropped by 10% of the maximum force.

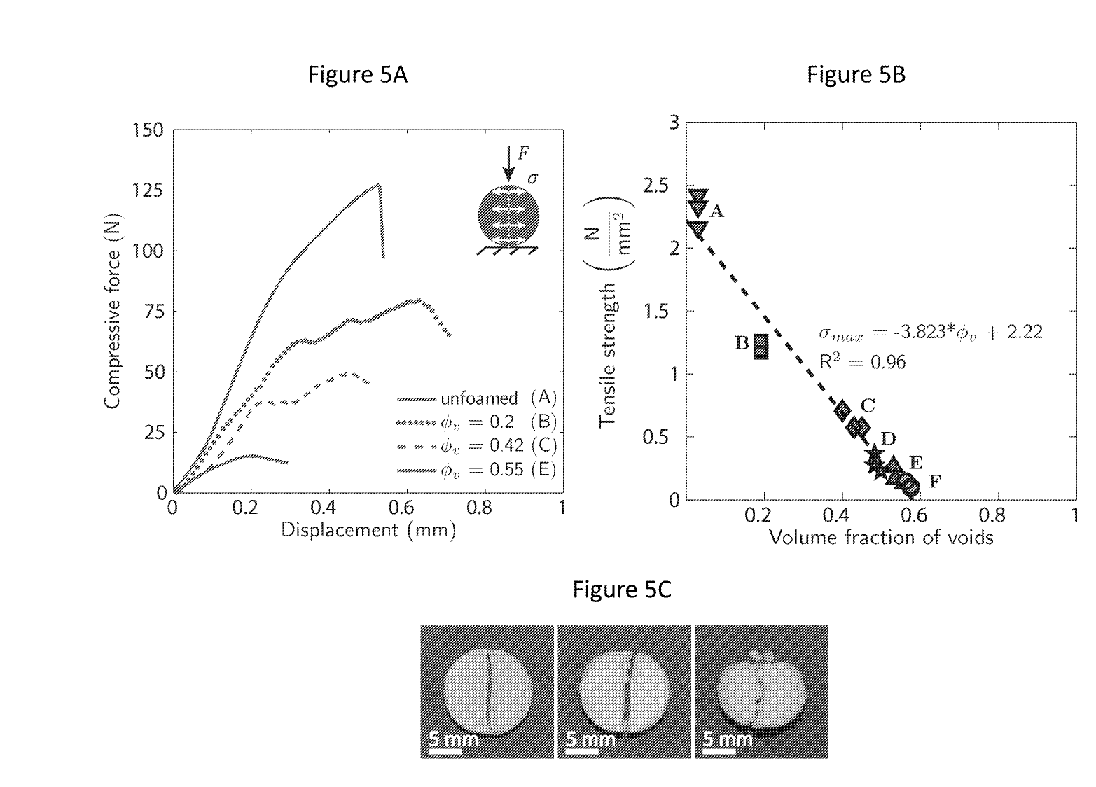

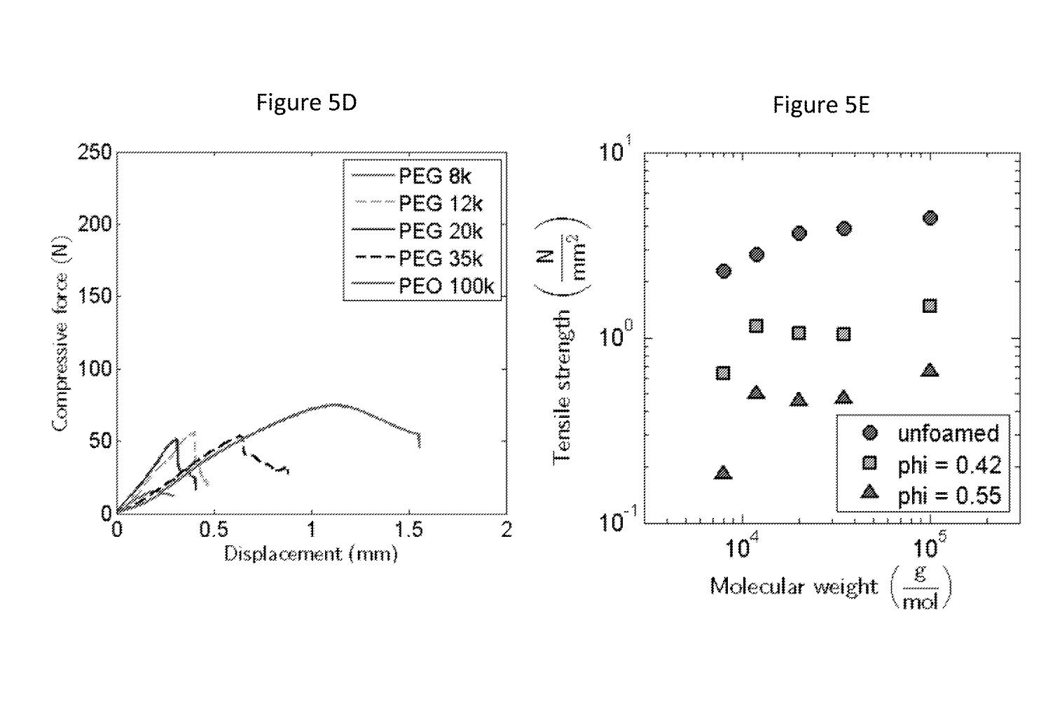

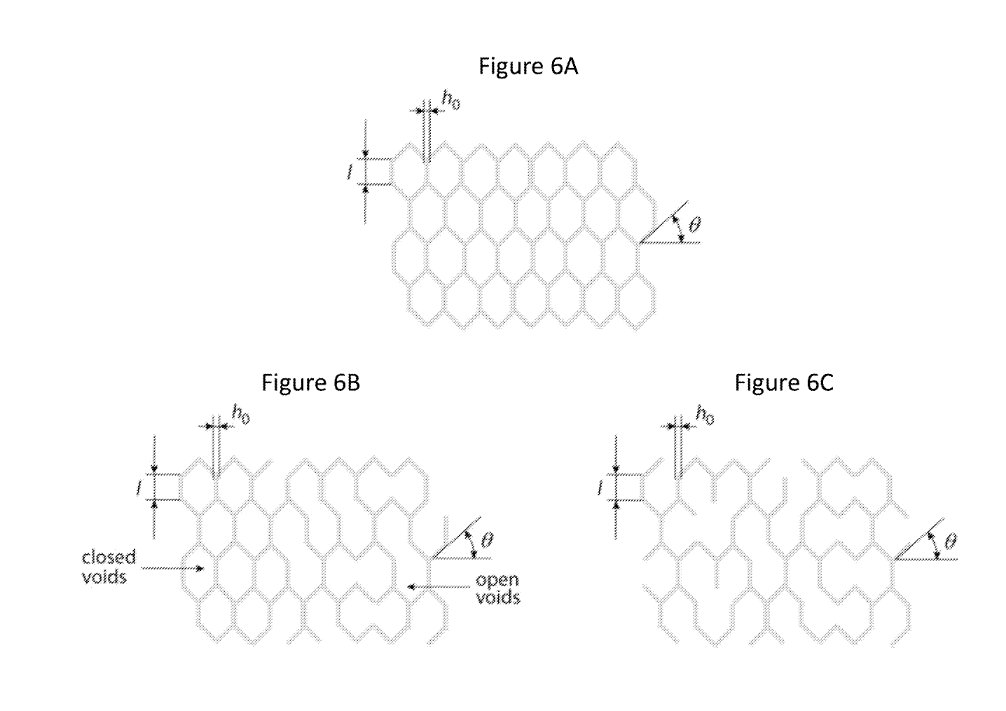

The force-displacement curves of the diametral compression test are shown in FIG. 5A. The curves are smooth at low displacements and reach a maximum as the displacement is increased. The experiment was stopped when the load dropped by 10% from the maximum. The samples fractured mostly in tension, which suggests that the maximum tensile stress can be calculated as

$${σ}_{max} = \frac{2 {F}_{max}}{π {D}_{0} {H}_{0}} \qquad ( 4 )$$

A plot of maximum or fracture strength, σ^{max} versus φ_{y} is shown in FIG. 5B, where the data for Fₘₐₓ are extracted from the force-displacement curves tabulated in Table 1. The σₘₐₓ decreases as the volume fraction of voids is increased. The decrease of the tensile stress is due to the reduced load-bearing area of the cellular material, as well as stress concentration around the voids.

Table 2 below illustrates excipients and process conditions of cellular dosage forms, and resulting dissolution times as obtained by the method presented in Example 3A, maximum compressive forces and tensile strengths.

TABLE 2

| | | | | | Properties | | |
|---|---|---|---|---|---|---|---|
| | Microstructural and Process Parameters | | | | | Maximum | |
| | Volume | | | | | compressive | Tensile |
| | faction of | | | | | force | strength |
| Excipient | void | Tₛ (° C.) | pₛ (MPa) | tᵣ (s) | t_{0.8} | (N) | (N/mm²) |
| Peg 8k | 0.03 | — | — | — | 28.54 | 121.2 | 2.3 |
| Peg 8k | 0.2 | 70 | 4.1 | 3 | 21.8 | 76.25 | |
| Peg 8k | 0.42 | 110 | 5.5 | 3 | 6.21 | 54.88 | 0.64 |
| Peg 8k | 0.49 | 130 | 4.1 | 60 | 3.37 | 28.81 | |
| Peg 8k | 0.55 | 130 | 6.2 | 3 | 2.3 | 20.81 | 0.183 |
| Peg 8k | 0.57 | 130 | 6.9 | 60 | 3.61 | 13.91 | |
| Peg 12k | <0.05 | — | — | — | 30.54 | 148.7 | 2.82 |
| Peg 12k | 0.42 | 110 | 5.5 | 3 | 15.95 | 101.3 | 1.15 |
| Peg 12k | 0.55 | 130 | 8.3 | 3 | 3.78 | 56.4 | 0.497 |
| Peg 20k | 0.05 | — | — | — | 32.83 | 192.3 | 3.65 |
| Peg 20k | 0.42 | 110 | 5.5 | 3 | 22.9 | 93.05 | 1.057 |
| Peg 20k | 0.55 | 130 | 8.3 | 3 | 5.18 | 51.57 | 0.455 |
| Peg 35k | 0.05 | — | — | — | 38.08 | 204.3 | 3.88 |
| Peg 35k | 0.42 | 110 | 5.5 | 3 | 27.88 | 91.08 | 1.03 |
| Peg 35k | 0.55 | 130 | 8.3 | 3 | 6.63 | 53.67 | 0.473 |
| Peo 100k | 0.05 | — | — | — | 61.3 | 231.5 | 4.4 |
| Peo 100k | 0.42 | 110 | 5.5 | 3 | 53.54 | 130.1 | 1.48 |
| Peo 100k | 0.55 | 130 | 8.3 | 3 | 33.43 | 74.83 | 0.66 |

Composition of the dosage form: 60% API (Acetaminophen) + 40% excipient. Amount of API in the sample: 245 mg. Nominal dimensions of disk specimens: diameter = 13 mm and thickness proportional to the void volume fraction (H₀ = 2.5 mm if φᵥ = 0). t_{0.8} is the time taken to release 80% (196 mg) of the drug present in the dosage form.

### Example 5

### Characterization of Excipients

This example demonstrates exemplary properties of excipients used for cellular dosage forms.

Diffusivity of the Dissolution Fluid into the Excipient and the Formulation:

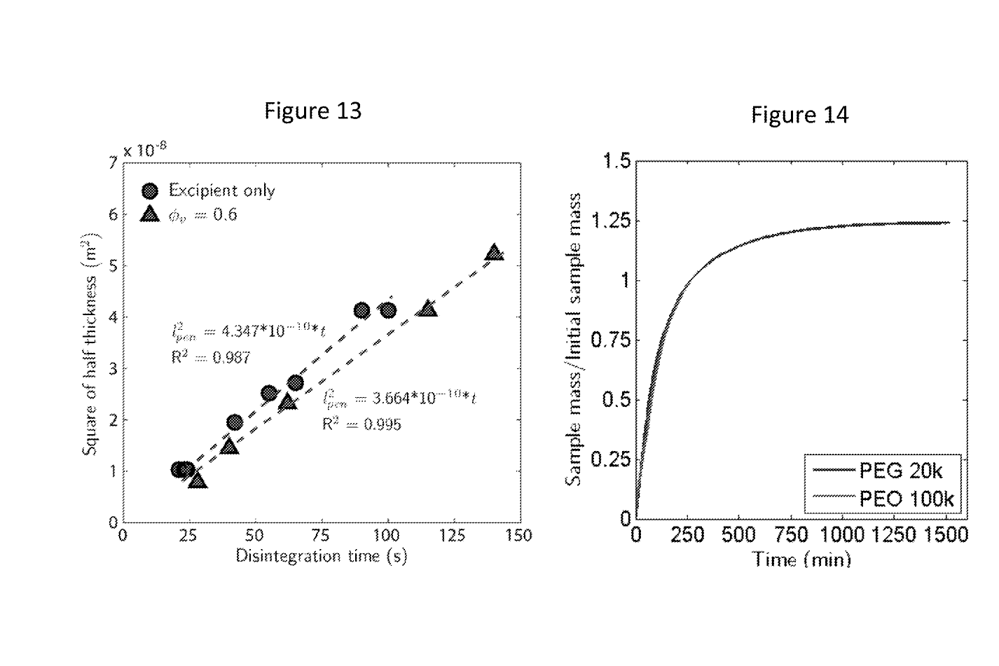

Cast (minimally porous) films of a given thickness were placed on a ring in a still dissolution medium at 37° C. and the time for the film to break apart was recorded. The results were plotted in a graph of square of half thickness of the film versus disintegration time, and the slope of the curve represented the effective diffusivity (according to t=lₚₑₙ²/D). The calculated effective diffusivity is 4.33×10⁻¹⁰ m²/s for the system with only the PEG 8000 excipient, and 3.67×10⁻¹⁰ m²/s for the excipient-drug system with a drug volume fraction of 0.6. Further, lₚₑₙ is assumed here to be equal to half of the thickness of the film. The results are shown in FIG. 13.

The average velocity at which the fluid front advances into the solid or the diffusivity of the dissolution medium in the formulation may also be determined by spectral methods. In this case, one side of the film is exposed to the dissolution medium. On the other side of the film, the concentration of dissolution fluid is monitored. As soon as the concentration of dissolution fluid raises substantially, the film is penetrated. This method is better suited for materials that have some mechanical strength (i.e., increased viscosity) after they are penetrated by the dissolution fluid.

### Sorption Tests to Determine the Amount of Water Sorbed by the Excipient at Equilibrium:

A dry sample of 10 mg was placed in a dynamic vapor sorption system. The sample was exposed to 95 percent humidity at 37° C. and the mass of the sample was monitored versus time. From the sample mass at equilibrium and the initial sample mass, the amount of water sorbed can be calculated. The results are shown in FIG. 14.

### Viscosity of the Excipient at Equilibrium Swelling:

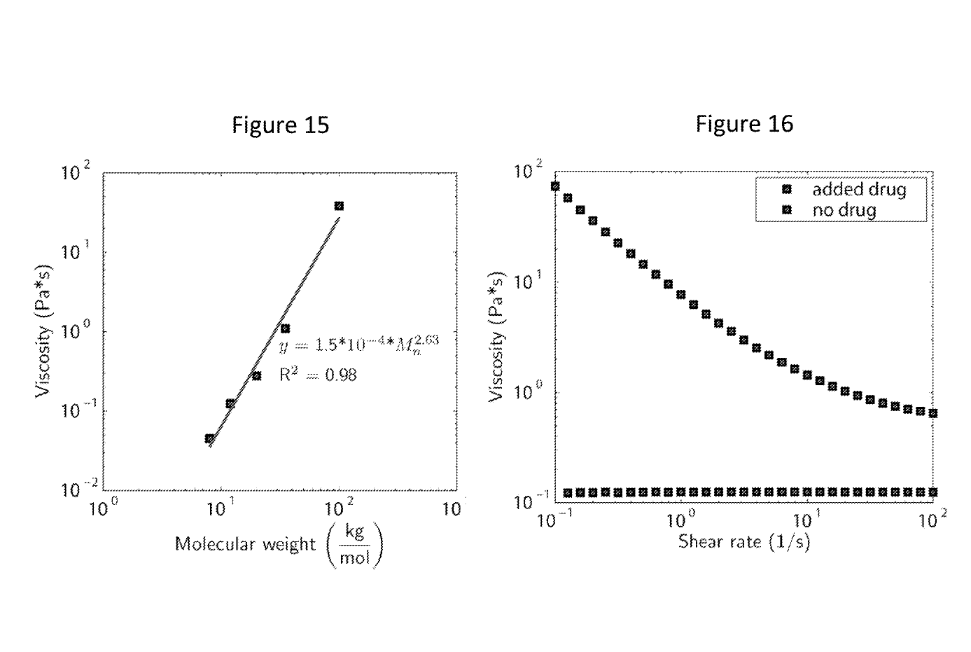

Polyethylene glycol powder was mixed with dissolution fluid. The mass of polymer was 0.5 times that of the fluid. The viscosity was measured by shear rheometry at a shear rate between 0.1 s⁻¹ and 100 s⁻¹ at a temperature of 37° C. The measured values for the viscosity were averaged over the entire range of shear rates. The results are shown in FIGS. 15 and 16.

### Solid-Liquid Interface Concentration:

Rotating disk experiments were conducted to estimate the concentration of the eroding polymer, c₀, at the solid-liquid interface. If it is assumed that the dissolution medium is a dilute solution and behaves as a Newtonian viscous fluid, the flux of the polymer eroding from a flat rotating surface can be expressed, provided the concentration boundary layer is at steady-state, by Levich's equation as:

$$j = 0.62 {( \frac{{ρ}_{f}}{{μ}_{f}} )}^{\frac{1}{6}} {D}^{\frac{2}{3}} {c}_{0} {Ω}^{\frac{1}{2}} \qquad ( 5 )$$

where ρ_{f} is the density of the dissolution medium, μ_{f} the viscosity, D the diffusivity of the polymer in the dissolution medium, and Ω the angular velocity.

$${c}_{0} = 1.61 {( \frac{{μ}_{f}}{{ρ}_{f}} )}^{\frac{1}{6}} {D}^{\frac{2}{3}} {Ω}^{\frac{1}{2}} j \qquad ( 6 )$$

All the parameters on the right side of Equation (6), except j, can be either estimated or calculated. Therefore, the average flux, j, in a rotating disk experiment is:

$$j = 0.8 \frac{{ρ}_{s} {H}_{0}}{{t}_{0.8}} \qquad ( 7 )$$

where ρₛ is the density of the eroding material, H₀ the initial thickness of the disk, and t_{0.8} the time taken to erode 80% of the sample. For ρₛ and H₀ can be calculated or estimated, t_{0.8} is the only parameter that needs to be found experimentally in order to derive j by Equation (7) and c₀ by Equation (6).

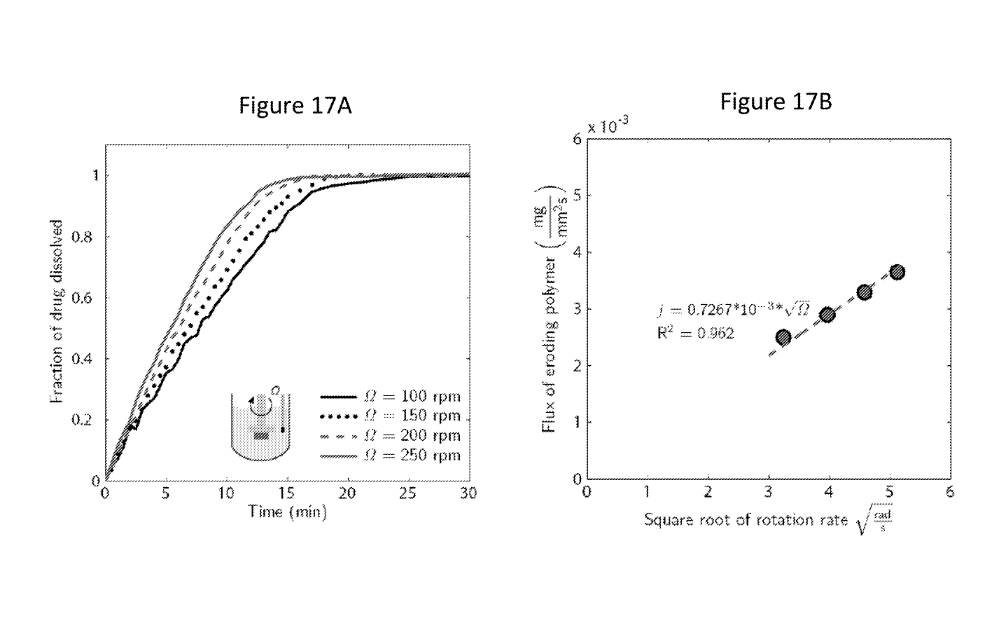

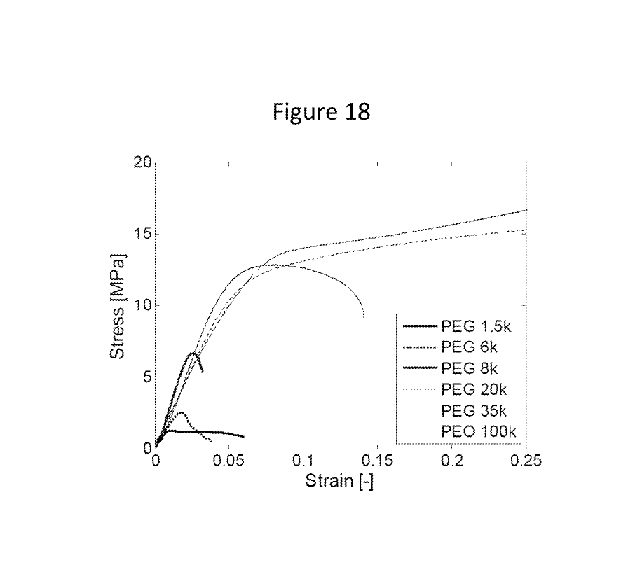

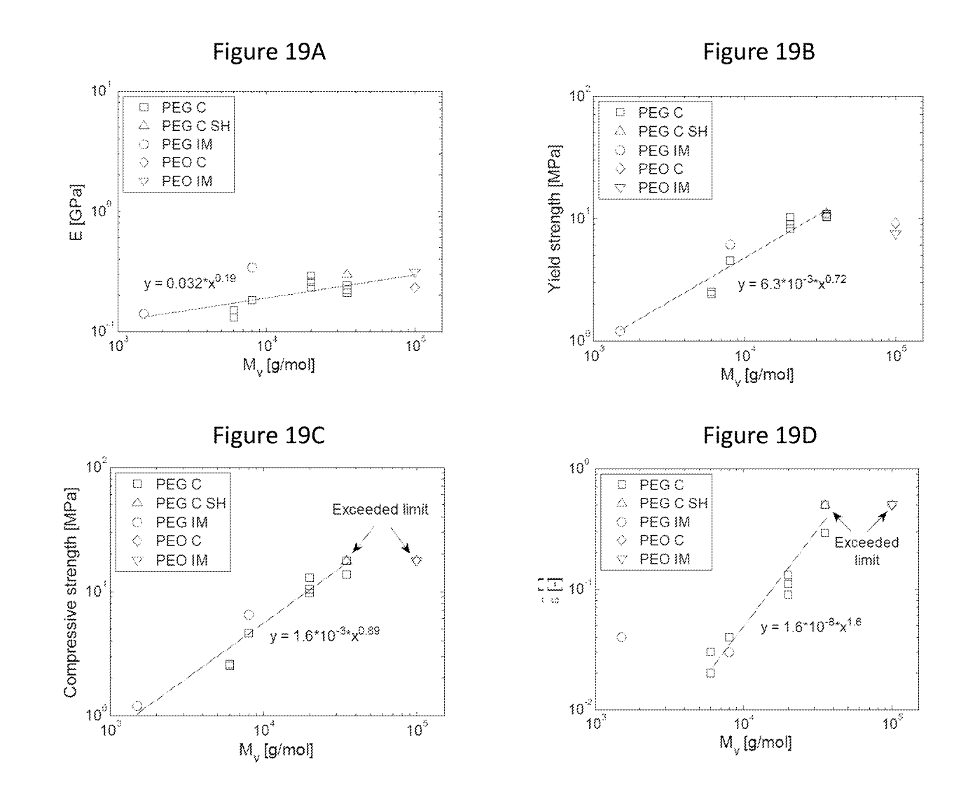

Rotating disk experiments were conducted at a temperature equal to 37° C. using dissolution medium according to the United States Pharmacopeia (USP) to determine t_{0.8}. The experiments were performed by attaching a 2.2 mm thick solid dosage form, with an excipient mass fraction of 0.95 and a drug mass fraction of 0.05 to the end of a rotating cylinder, and measuring the amount of drug released as a function of time at a given angular velocity. A plot of the fraction of drug released versus time at various angular velocities is shown in FIG. 17. The dissolution time is decreased as the rotation rate is increased. The respective values obtained for t_{0.8} derived from FIG. 17 are inserted into Equation (7), and the values so obtained for j are plotted versus the square root of the angular velocity in FIG. 18. The data of j versus Ω^{0.5} can be fitted to a straight line as j=0.7267 Ω^{0.5}, suggesting that Equation (6) is a reasonable approximation for calculating the flux of the eroding polymer within the range of parameters applied. Using μ_{f}=0.001 Pa·s, ρ_{f}=1000 kg/m³, D=9.81×10⁻¹¹ m²/s, and j=0.7267 Ω^{0.5}, estimated c₀ by Equation (6) is 551 kg/m³.

### Mechanical Properties of the Solid Excipient:

Samples for compression tests were prepared by either hot melt casting or injection molding. Compression tests were performed on pure PEG and PEO specimen. The ASTM standard test method for compressive properties of rigid plastics, ASTM D695-10, was used as the protocol to execute compression tests. The testing machine was a Zwick Roell Z2.5 with a 2.5 kN load cell (Zwick GmbH & Co. KG, Ulm, Germany), equipped with compression platens. A speed of 1.3 mm/min was applied for the platens to move relatively towards each other. Tables 3 and 4 summarize the parameters applied to execute compression tests.

Table 3 below illustrates material, geometric, and process parameter values applied for sample preparation erosion and dissolution tests. Erosion test samples were casted, whereas dissolution test samples were injection-molded (IM). Aspirin was used as API.

TABLE 3

| | Compression | Compression | Nano- |
|---|---|---|---|
| | test (Casted | test (IM | indentation |
| Parameter | sample) | sample) | (IM sample) |
| Materials | PEG or PEO | PEG or PEO | KCIR & Mann., |
| | | | others^{a} |
| Diameter (mm) | 12.7 | 12.7 | 12.7 |
| Thickness (mm) | 18 | 23 | 2 |
| Melt Temperature (° C.) | 90 | 75 | 185, 75, 170^{b} |
| Mold Temperature (° C.) | 25 | 25 | 25 |
| Injection Flow Rate | — | 5 | 5 |
| (cm³/s) | | | |
| Hold Pressure (MPa) | — | 40 | 100 |
| Casting pressure (MPa) | 15 | — | — |
| Cooling Time (s) | 60 | 60 | 30 |

^{a}“Others” refers to the excipients PEO 100k, and 75% Eudragit L100-55 25% Triethylcitrate
^{b}The melt temperature of the Kollicoat IR - Mannitol sample was 185° C., the melt temperature of PEO 100k 75° C., and the melt temperature of 75% Eudragit L100-55 25% Triethylcitrate was 170° C.

Table 4 below illustrates Data of mechanical properties of PEG and PEO from compression tests.

TABLE 4

| | Molecular | Young's | Yield | Compressive | Strain at |
|---|---|---|---|---|---|
| | weight | modulus | strength | strength | fracture |
| Material | (g/mol) | (GPa) | (MPa) | (MPa) | (—) |
| PEG 1.5k^{a} | 1,500 | 0.14 | 1.2 | 1.2 | 0.04 |
| PEG 6k^{b} | 6,000 | 0.13 | 2.5 | 2.5 | 0.03 |
| PEG 6k^{b} | 6,000 | 0.15 | 2.5 | 2.5 | 0.02 |
| PEG 8k^{a} | 8,000 | 0.34 | 6.1 | 6.5 | 0.03 |
| PEG 8k^{b} | 8,000 | 0.18 | 4.5 | 4.6 | 0.04 |
| PEG 20k^{b} | 20,000 | 0.23 | 9.0 | 10.4 | 0.09 |
| PEG 20k^{b} | 20,000 | 0.26 | 8.2 | 9.6 | 0.11 |
| PEG 20k^{b} | 20,000 | 0.29 | 10.2 | 12.8 | 0.13 |
| PEG 35k^{b} | 35,000 | 0.22 | 10.2 | >17.6 | >0.5 |
| PEG 35k^{b} | 35,000 | 0.21 | 10.8 | >17.6 | >0.5 |
| PEG 35k^{b} | 35,000 | 0.24 | 10.4 | 13.6 | 0.29 |
| PEG 35k^{c} | 35,000 | 0.30 | 11.0 | >17.6 | >0.5 |
| PEO 100k^{a} | 100,000 | 0.31 | 7.5 | >17.6 | >0.5 |
| PEO 100k^{b} | 100,000 | 0.23 | 9.1 | >17.6 | >0.5 |
| Mannitol^{d} | 182 | — | — | 0.8 | <0.02 |

^{a}Based on injection-molded sample
^{b}Based on casted sample
^{c}Based on strain-hardened casted sample
^{d}Based on compression-molded sample. The material could not be manufactured defect-free and appropriately tested for Young's modulus and Yield strength.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

1. A pharmaceutical solid dosage form comprising one or more hydrophilic excipients and one or more active ingredients, wherein the dosage form has a cellular microstructure comprising a plurality of cells filled with a gas that is non-reactive with the active ingredients and the excipients, and having solid cell walls comprising the one or more active ingredients and the one or more excipients,
wherein:
(a) a fraction of the total number of cells in the solid dosage form are part of a cluster of two or more interconnected cells, said fraction being in a range from 0.3 to 1;
(b) the cells have average size in a range from 1 μm to 1500 μm;
(c) the cells have average wall thickness, h₀, not greater than 500 μm;
(d) the solid dosage form has void cell volume fraction with respect to total volume, φᵥ, in a range from 0.2 to 0.85; and
(e) the solid dosage form has at least one dimension greater than 1 mm.

2. The dosage form of claim 1, wherein standard deviation of the cell size is less than the average cell size in the solid dosage form.

3. The dosage form of claim 1, wherein standard deviation of the cell wall thickness is less than the average cell wall thickness.

4. The dosage form of claim 1, wherein the one or more excipients is/are absorptive of a physiological fluid under physiological conditions when the one or more excipients is/are exposed to the physiological fluid and wherein rate of penetration of the physiological fluid into the solid dosage form is greater than about h₀/1800 μm/s.

5. The dosage form of claim 4, wherein the solid dosage form has a composition and structure such that effective diffusion coefficient of the physiological fluid into the solid is no less than 1-10⁻¹¹ m²/s.

6. The dosage form of claim 1, wherein shear viscosity of the one or more excipients is no greater than about 200 Pa·s upon absorption of a physiological fluid.

7. The dosage form of claim 1, wherein solubility of the excipient in a physiological fluid is no less than about 1 g/l.

8. The dosage form of claim 1, wherein tensile strength of the dosage form is no less than about 0.05 N/mm².

9. The dosage form of claim 1, wherein the one or more excipients comprises a polymer having weight average molecular weight in a range from 1,000 g/mol to 300,000 g/mol.

10. The dosage form of claim 1, wherein the one or more excipients comprises polyethylene glycol (PEG) having weight average molecular weight in a range from 1,500 g/mol to 200,000 g/mol.

11. The dosage form of claim 1, wherein the solid cell walls of the dosage form are composed of a non-porous solid having void volume fraction no greater than about 0.1.

12. The dosage form of claim 1, wherein the cell walls of the dosage form have an excipient volume fraction, with respect to total cell wall volume, greater than 0.05.

13. The dosage form of claim 1, further comprising one or more fast eroding excipients wherein each of the one or more fast eroding excipients has a characteristic erosion rate (ψ=(solubility×diffusivity^{1/2})/(π^{1/2}×density)) greater than about 5×10⁻⁵ m/s^{1/2} upon ingestion by the subject, wherein volume fraction of the fast eroding excipient(s) with respect to the total wall volume (φₑ), is within a range from about 0.03 to about 0.4.

14. The dosage form of claim 1, further comprising one or more effervescent agents, wherein volume fraction of the effervescent agent(s) with respect to total wall volume (ψ_{ee,}) is within a range from about 0.03 to about 0.4.

15. The dosage form of claim 1, further comprising one or more fillers, one or more stabilizers, one or more preservatives, one or more taste maskers, one or more colorants, or any combination thereof.

16. The dosage form of claim 1, wherein solid drug contents of the dosage form are converted into molecularly dissolved units in less than about 30 minutes after ingestion.

17. A method of manufacturing a pharmaceutical cellular dosage form, the method comprising:
(a) mixing components (i) and components (ii) with application of shear force:
wherein components (i) comprise one or more excipients,
wherein components (ii) comprise one or more pharmaceutically active ingredients;
(b) introducing a foaming agent and/or a supercritical fluid under pressure, into the mixture; and
(c) introducing the mixture into a mold,
wherein the pharmaceutical cellular dosage form produced thereby has a cellular microstructure comprising a plurality of cells filled with a gas that is non-reactive with the active ingredients and the excipients and solid cell walls comprising the one or more active ingredients and the one or more excipients, wherein one, two, three, four, or all five of items (A) through (E) apply:
(A) a fraction of the total number of cells in the solid dosage form are part of a cluster of two or more interconnected cells, said fraction being in a range from 0.3 to 1;
(B) the cells have average size in a range from 1 μm to 1500 μm;
(C) the cells have average wall thickness, h₀, not greater than 500 μm;
(D) the solid dosage form has void volume fraction with respect to total volume, φᵥ, in a range from 0.2 to 0.85; and
(E) the solid dosage form has at least one dimension greater than 1 mm.

18. The method of claim 17, further comprising
dissolving the foaming agent in the mixture under shear force so that the concentration of the foaming agent is homogeneous in the mixture.

19. The method of claim 17, further comprising
reducing the pressure of the mixture so that the foaming agent is supersaturated in the mixture and gas bubbles nucleate and grow.

20. The method of any claim 17, further comprising
reducing the temperature of the mixture so that the mixture solidifies as the cellular dosage forms.

21. The method of claim 17, further comprising
introducing a coating material in the mold or applying the coating material directly to the dosage form.

22. The dosage form of claim 1, wherein the cells comprise voids of substantially convex shape filled with a gas comprising one or more of N₂, CO₂, or air.
